SEQUENCE LISTING (5 sequences)

SEQ ID NO: 1 - protein, 518 aa, Aplysia punctata
MDGVSRNRRQCNREVCGSTYDVAVVGAGPGGANSAYMLRDSGLDIAVFEYSDRVGGRLFTYQLPNTPDVNLEIGGMRFIEGAMHRLWRVISELGLTPKVFKEGFGKEGRQRFYLRGQSLTKKQVKSGDVPYDLSPEEKENQGNLVEYYLEKLTGLQLNGEPLKREVALKLTVPDGRFLYDLSFDEAMDLVASPEGKEFTRDTHVFTGEVTLDASAVSLFDDHLGEDYYGSEIYTLKEGLSSVPQGLLQAFLDAADSNEFYPNSHLKALRRKTNGQYVLYFEPTTSKDGQTTINYLEPLQVVCAQRVILAMPVYALNQLDWNQLRNDRATQAYAAVRPIPASKVFMTFDQPWWLENERKSWVTKSDALFSQMYDWQKSEASGDYILIASYADGLKAQYLRELKNQGEDIPGSDPGYNQVTEPLKDTILDHLTEAYGVERDSIPEPVTAASQFWTDYPFGCGWITWRAGFHFDDVISTMRRPSLKDEVYVVGADYSWGLISSWIEGALETSENVINDYFL

SEQ ID NO: 2 - protein, 64 aa, synthetic construct
VDNKFNKEMRNAYWEIALLPNLNNQQKRAFIRSLYDDPSQSANLLAEAKKLNDAQAPKGGSGGS

SEQ ID NO: 3 - protein, 64 aa, synthetic construct
VDNRFNAEMRNAYWEIALLPNLNNQQRRAFIRSLYDDPSQSANLLAEARALNDAQAPAGGSGGS

SEQ ID NO: 4 - protein, 589 aa, synthetic construct
MVDNRFNAEMRNAYWEIALLPNLNNQQRRAFIRSLYDDPSQSANLLAEARALNDAQAPAGGGSGGGDGGGSMDGVSRNRRQCNREVCGSTYDVAVVGAGPGGANSAYMLRDSGLDIAVFEYSDRVGGRLFTYQLPNTPDVNLEIGGMRFIEGAMHRLWRVISELGLTPKVFKEGFGKEGRQRFYLRGQSLTKKQVKSGDVPYDLSPEEKENQGNLVEYYLEKLTGLQLNGEPLKREVALKLTVPDGRFLYDLSFDEAMDLVASPEGKEFTRDTHVFTGEVTLDASAVSLFDDHLGEDYYGSEIYTLKEGLSSVPQGLLQAFLDAADSNEFYPNSHLKALRRKTNGQYVLYFEPTTSKDGQTTINYLEPLQVVCAQRVILAMPVYALNQLDWNQLRNDRATQAYAAVRPIPASKVFMTFDQPWWLENERKSWVTKSDALFSQMYDWQKSEASGDYILIASYADGLKAQYLRELKNQGEDIPGSDPGYNQVTEPLKDTILDHLTEAYGVERDSIPEPVTAASQFWTDYPFGCGWITWRAGFHFDDVISTMRRPSLKDEVYVVGADYSWGLISSWIEGALETSENVINDYFL

SEQ ID NO: 5 - protein, 589 aa, synthetic construct; identical to SEQ ID NO: 4

(19) United States
(12) Patent Application Publication
(10) Pub. No.: US 2026/0207715 A1
(43) Pub. Date: Jul. 23, 2026

(54) KIT FOR COMBINATION THERAPY OF TUMOR DISEASES AND/OR INFECTIONS

(71) Applicant: JULIUS-MAXIMILIANS-UNIVERSITÄT WÜRZBURG, Würzburg (DE)

(72) Inventors: Martina RASCHIG, Würzburg (DE); Alena WOLKERSDORFER, Bonn (DE); Lukas HAHN, Sulzbach am Main (DE); Lorenz MEINEL, Würzburg (DE); Tessa LÜHMANN, Würzburg (DE)

(21) Appl. No.: 19/138,890

(22) PCT Filed: Dec. 14, 2023
(86) PCT No.: PCT/EP2023/085929

§ 371 Date: Jun. 13, 2025

(30) Foreign Application Priority Data
Dec. 16, 2022 (EP) 22214262.2

(51) Int. Cl. A61K 38/44; A61K 38/17; A61K 47/54; A61K 47/60

(52) U.S. Cl. CPC A61K 38/44; A61K 38/179; A61K 47/545; A61K 47/60

(57) ABSTRACT

The present invention relates to a kit of active pharmaceutical ingredients comprising (1) a fusion component comprising an agent, which increases the level of reactive oxygen species (ROS), such as Aplysia punctata ink toxin (APIT), and a targeting sequence, and (2) a prodrug or a proreporter comprising phenylboronic acid or phenylboronic ester derivatives. The present invention relates to said kit of pharmaceutical compositions for use in the diagnosis, prevention and/or treatment of proliferative diseases, viral and/or non-viral infections. The present invention further relates to a method of diagnosis, prevention and/or treatment of a proliferative disease, a viral infection and/or a non-viral infection.

The present invention relates to a kit of active pharmaceutical ingredients comprising (1) a fusion component comprising an agent, which increases the level of reactive oxygen species (ROS), such as Aplysia punctata ink toxin (APIT), and a targeting sequence, and (2) a prodrug or a proreporter comprising phenylboronic acid or phenylboronic ester derivatives. The present invention relates to said kit of pharmaceutical compositions for use in the diagnosis, prevention and/or treatment of proliferative diseases, viral and/or non-viral infections. The present invention further relates to a method of diagnosis, prevention and/or treatment of a proliferative disease, a viral infection and/or a non-viral infection.

### BACKGROUND OF THE INVENTION

In the treatment of almost all diseases, such as cancer and chronic inflammation, the aim is to achieve therapeutic success with as few side effects as possible. Therefore, a combination of reliable diagnostic tools and specific medication is the basis of any therapy. In the last decades, so-called “smart systems” have been investigated in the fields of biomedicine and nanoscience, where the release of the drug is controlled by different triggers, in particular pH, temperature, redox reactions, (bio) chemical reactions and so on (“prodrug” strategy). In addition, many studies have been conducted that address targeted, site-specific delivery (“targeting”) of drugs. Almost all these systems have specific limitations, including safety, efficacy, and selectivity, so new approaches will need to be developed in the future.

In recent years, the role of reactive oxygen species (ROS) has been highlighted, acting as regulators of various cellular functions (Sies and Jones, 2020). Increases in ROS are observed in various pathological conditions such as tumor promotion and progression (Perillo et al., 2020), Huntington's disease (Johri and Beal, 2012), Alzheimer's disease (Mattson, 2004), and Parkinson's disease (Exner et al., 2012). Moreover, it is known that highly elevated levels of ROS can trigger programmed cell death (Reczek and Chandel, 2017). As a result, cancer treatment strategies based on modulation of ROS levels have been described (Perillo et al., 2020) (as shown in FIG. 1A).

In addition, the intrinsic ROS gradient between healthy and diseased tissues can be used to control drug release (Peiró Cadahía et al., 2019) (see FIG. 1B). However, these strategies are critical because the ROS gradient is often not significantly mature, leading to uncontrolled hydrolysis and release of the drugs, thus providing insufficient selectivity.

There is a need in the art for improved means and methods for the treatment of cancer which provide improved selectivity and/or effectivity.

### SUMMARY OF THE INVENTION

According to the present invention this object is solved by a kit of active pharmaceutical ingredients comprising
(1) a fusion component comprising an agent, which increases the level of reactive oxygen species (ROS), and a targeting sequence, and
(2) a prodrug or proreporter comprising a compound of formula I or formula II

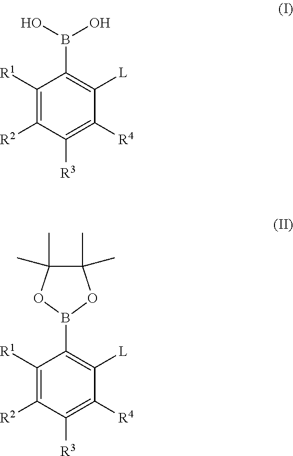

wherein
R¹ to R⁴ are each independently selected from H, OR⁵, NHR⁵, NHC(O)R⁵, SR⁵, halogen, C₁-C₆ alkyl, C₁-C₆ alkoxy, aryl, and C₁-C₂₀ alkyloxycarbonyl,
R⁵ is selected from H, C₁-C₆ alkyl, C₁-C₆ alkoxy, and C₁-C₆ alkyl or C₁-C₆ alkoxy substituted with a reactive group for attachment of a polymer, peptide or protein,
L is selected from —(CH₂)₂—C(O)O—X and —CH═CH—C(O)O—X, and
X is a drug or a reporter,
or a pharmaceutically acceptable salt thereof.

According to the present invention this object is solved by kit of active pharmaceutical ingredients of the present invention for use in the diagnosis, prevention and/or treatment of proliferative diseases, viral infections and/or non-viral infections.

According to the present invention this object is solved by a method for the diagnosis, prevention and/or treatment of a proliferative disease, a viral infection and/or a non-viral infection, comprising the administration of therapeutically effective amounts of the kit of active pharmaceutical ingredients of the present invention to a subject in need thereof.

### DESCRIPTION OF THE PREFERRED EMBODIMENTS OF THE INVENTION

Before the present invention is described in more detail below, it is to be understood that this invention is not limited to the particular methodology, protocols and reagents described herein as these may vary. It is also to be understood that the terminology used herein is for the purpose of describing particular embodiments only and is not intended to limit the scope of the present invention which will be limited only by the appended claims. Unless defined otherwise, all technical and scientific terms used herein have the same meanings as commonly understood by one of ordinary skill in the art. For the purpose of the present invention, all references cited herein are incorporated by reference in their entireties.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “0.5 to 10” should be interpreted to include not only the explicitly recited values of 0.5 to 10, but also include individual values and sub-ranges within the indicated range. Thus, included in this numerical range are individual values such as 0.5, 1, 1.5, 2, 2.5, 3, 4, 5 . . . 8, 8.5, 9, 9.5, 10 and sub-ranges such as from 2 to 8, 4 to 5, etc. This same principle applies to ranges reciting only one numerical value, such as “less than 1 mm”. Furthermore, such an interpretation should apply regardless of the breadth of the range or the characteristics being described.

### Kits of Active Pharmaceutical Ingredients

As outlined above, the present invention provides a kit of active pharmaceutical ingredients. Said kit of active pharmaceutical ingredients comprises
(1) a fusion component comprising an agent, which increases the level of reactive oxygen species (ROS), and a targeting sequence, and
(2) a prodrug or proreporter comprising a compound of formula I or formula II

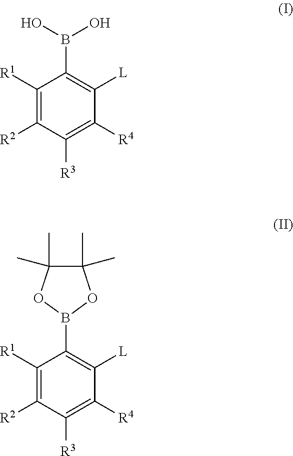

wherein
R¹ to R⁴ are each independently selected from H, OR⁵, NHR⁵, NHC(O)R⁵, SR⁵, halogen, C₁-C₆ alkyl, C₁-C₆ alkoxy, aryl, and C₁-C₂₀ alkyloxycarbonyl,
R⁵ is selected from H, C₁-C₆ alkyl, C₁-C₆ alkoxy, and C₁-C₆ alkyl or C₁-C₆ alkoxy substituted with a reactive group for attachment of a polymer, peptide or protein,
L is selected from —(CH₂)₂—C(O)O—X and —CH═CH—C(O)O—X, and
X is a drug or a reporter,
or a pharmaceutically acceptable salt thereof.

Active ingredients are the substances in drugs that are responsible for the health effects/benefits experienced by the subjects. The active ingredient in a pharmaceutical drug is called an active pharmaceutical ingredient (API).

The kit of the present invention can further comprise pharmaceutically acceptable expedient(s) and/or carrier.

Said active pharmaceutical ingredients (1) and (2) are each provided as a pharmaceutical composition, i.e. as a pharmaceutical composition in itself.

They can further comprise pharmaceutically acceptable expedient(s) and/or carrier.

The kit of active pharmaceutical ingredients of the present invention can also be considered as a kit of parts, wherein the parts are the active pharmaceutical ingredients or their respective pharmaceutical composition.

The kit of the present invention can also be considered as a collection of the two active pharmaceutical ingredients (1) and (2).

### Active Pharmaceutical Ingredient (1)—Fusion Component

The active pharmaceutical ingredient (1) of the kit of the present invention is a fusion component comprising
an agent, which increases the level of reactive oxygen species (ROS), and
a targeting sequence.

The active pharmaceutical ingredient (1) has the task of selectively generating oxidative stress and the associated increase in ROS molecules at the site of action.

An agent, which increases the level of reactive oxygen species (ROS), can be selected from: beta-lapachone (ARQ 501), NOV-002 (a mimetic of glutathione disulfide), gamitrinib, bortezomib, celecoxib, imatinib, erlotinib, vermurafenib, rituximab, procarbazine, doxorubicin, arsenic trioxide, 2-methoxyestradiol, N-(4-hydroxyphenyl retinamide), platin agents, elesclomol (STA-4783), sanguinarine, rapamycin, D-amphetamine, and ferrocene und its derivates.

Preferably, the agent, which increases the level of reactive oxygen species (ROS), is a protein, more preferably an enzyme.

Examples for enzymes, which increase the level of reactive oxygen species (ROS), are glucose oxidases, amino acid oxidases, format oxidases, NADH oxidases, NADPH oxidases and nucleoside oxidases.

In one embodiment, the enzyme which increases the level of reactive oxygen species (ROS), is an L-amino acid oxidase (LAAO, EC 1.4.3.2). LAAO are highly specific towards different L-amino acids. LAAO are found in various species and show diverse physiological roles. LAAO are found primarily in various animal fluids, including serum, venoms, ink, mucus and mammary milk and tissue extract. So far, snake venom LAAO (SV-LAAO) are the most thoroughly investigated amino acid oxidases. function and bioactivity of LAAO include antibacterial and antiparasitic properties as a main defense of the organism or part of the immune defense. Said defense mechanism of LAAO can be primarily traced back to the generated H₂O₂.

In a preferred embodiment, the active pharmaceutical ingredient (1) of the kit of the present invention is a fusion protein comprising
Aplysia punctata ink toxin (APIT), and
a targeting sequence.

The Aplysia punctata ink toxin (APIT) comprises or consists of the amino acid sequence of SEQ ID NO. 1.

APIT is a FAD-binding L-amino acid oxidase (Butzke et al., 2005).

APIT has an antitumor effect which is based on the selective degradation of the essential L-amino acids L-lysine and L-arginine. This redox reaction produces as reactive byproducts hydrogen peroxide (H₂O₂) and NH₄, which are also detrimental to the tumor. See e.g. FIG. 2A.

According to the present invention, the protein, which increases the level of reactive oxygen species (ROS), such as APIT, also serves to increase the level or concentration of reactive oxygen species (ROS), such as H₂O₂, at the site of action, namely at the tumor site.

```
SEQ ID NO 1: amino acid number: 518, molecular weight: 58502.56 g/mol
  1 10 20 30 40 50 60
MDGVSRNRRQC NREVCGSTYD VAVVGAGPGG ANSAYMLRDS GLDIAVFEYS DRVGGRLFTY
                70 80 90 100 110 120
QLPNTPDVNL EIGGMRFIEG AMHRLWRVIS ELGLTPKVFK EGFGKEGRQR FYLRGQSLTK
              130 140 150 160 170 180
KQVKSGDVPY DLSPEEKENQ GNLVEYYLEK LTGLQLNGEP LKREVALKLT VPDGRFLYDL
              190 200 210 220 230 240
SFDEAMDLVA SPEGKEFTRD THVFTGEVTL DASAVSLFDD HLGEDYYGSE IYTLKEGLSS
              250 260 270 280 290 300
VPQGLLQAFL DAADSNEFYP NSHLKALRRK TNGQYVLYFE PTTSKDGQTT INYLEPLQVV
              310 320 330 340 350 360
CAQRVILAMP VYALNQLDWN QLRNDRATQA YAAVRPIPAS KVFMTFDQPW WLENERKSWV
              370 380 390 400 410 420
TKSDALFSQM YDWQKSEASG DYILIASYAD GLKAQYLREL KNQGEDIPGS DPGYNQVTEP
              430 440 450 460 470 480
LKDTILDHLT EAYGVERDSI PEPVTAASQF WTDYPFGCGW ITWGRAFHFD DVISTMRRPS
              490 500 510
LKDEVYVVGA DYSWGLISSW IEGALETSEN VINDYFL
```

The targeting sequence preferably targets cell types or tissues of interest, in particular cancer cells. The targeting sequence can also target viruses, bacterial cells or cells of other microorganisms.

In one embodiment, the targeting sequence targets cancer cells, such as human epidermal growth factor receptor (HER2)-overexpressing cells.

In other embodiments, the targeting sequence targets human immunodeficiency virus (HIV), hepatitis B virus (HBV) or/and hepatitis C virus (HCV).

The targeting sequence can be an antibody or an antibody fragment, a targeting peptide or peptide-like affibody, a nucleic acid or small targeting molecule.

Preferably, the active pharmaceutical ingredient (1) is a fusion protein, wherein the targeting sequence is preferably fused N-terminally to APIT.

In one embodiment, the active pharmaceutical ingredient (1) is a conjugate. Here, conjugation methods which are known in the art can be used to conjugate the targeting sequence to e.g.

APIT. For example, conjugation via N-terminal modification using Schiff base reaction+reductive amination. For example, conjugation via modification of amino acids, such as lysine, using active esters preferably NHS-esters.

In one preferred embodiment, the targeting sequence is a variant of an antibody, antibody fragment, targeting peptide, peptide-like affibody or small targeting molecule, wherein the lysine residues are deleted or substituted, preferably with arginine and/or alanine.

Said elimination of lysine residues by deletion or substitution preferably has a thermo-dynamically stabilizing effect. Said elimination of lysine residues can also have an effect on binding to a respective target receptor, such as it can be less interfering with a receptor binding site.

For example, in an embodiment where the targeting sequence targets human epidermal growth factor receptor (HER2)-overexpressing cells, said elimination of lysine residues results in less interfering with the Her2/neu receptor binding site.

In a preferred embodiment, the targeting sequence is zHER2:342. zHER2:342 comprises or consists of the amino acid sequence of SEQ ID NO. 2.

SEQ ID NO. 2:
VDNKFNKEMR NAYWEIALLP NLNNQQKRAF IRSLYDDPSQ
SANLLAEAKK LNDAQAPKGG SGGS

In one embodiment, the targeting sequence is a zHER2:342 variant wherein the lysine residues are deleted or substituted, preferably with arginine and/or alanine. In one embodiment, the targeting sequence is K4R/K7A/K27R/K49R/K50A/K58A zHER2:342.

K4R/K7A/K27R/K49R/K50A/K58A zHER2:342 comprises or consists of the amino acid sequence of SEQ ID NO. 3.

SEQ ID NO. 3:
VDNRFNAEMR NAYWEIALLP NLNNQQRRAF IRSLYDDPSQ
SANLLAEARA LNDAQAPAGG SGGS

Such zHER2:342 variant wherein the lysine residues are deleted or substituted is highly suitable to be further modified, such as by PEGylation.

In one embodiment, the fusion protein is zHER2:342-APIT.

zHER2:342-APIT comprises or consists of the amino acid sequence of SEQ ID NO. 4:

```
SEQ ID NO 4: amino acid number: 583, molecular weight: 65723.67 g/mol
  1 10 20 30 40 50 60
MVDNRFNAEMR NAYWEIALLP NLNNQQRRAF IRSLYDDPSQ SANLLAEARA LNDAQAPAGG
                70 80 90 100 110 120
GSGGGDGGGS MDGVSRNRRQ CNREVCGSTY DVAVVGAGPG GANSAYMLRD SGLDIAVFEY
              130 140 150 160 170 180
SDRVGGRLFT YQLPNTPDVN LEIGGMRFIE GAMHRLWRVI SELGLTPKVF KEGFGKEGRQ
              190 200 210 220 230 240
RFYLRGQSLT KKQVKSGDVP YDLSPEEKEN QGNLVEYYLE KLTGLQLNGE PLKREVALKL
              250 260 270 280 290 300
TVPDGRFLYD LSFDEAMDLV ASPEGKEFTR DTHVFTGEVT LDASAVSLFD DHLGEDYYGS
              310 320 330 340 350 360
EIYTLKEGLS SVPQGLLQAF LDAADSNEFY PNSHLKALRR KTNGQYVLYF EPTTSKDGQT
              370 380 390 400 410 420
TINYLEPLQV VCAQRVILAM PVYALNQLDW NQLRNDRATQ AYAAVRPIPA SKVFMTFDQP
              430 440 450 460 470 480
WWLENERKSW VTKSDALFSQ MYDWQKSEAS GDYILIASYA DGLKAQYLRE LKNQGEDIPG
              490 500 510 520 530 540
SDPGYNQVTE PLKDTILDHL TEAYGVERDS IPEPVTAASQ FWTDYPFGCG WITWRAGFHF
              550 560 570 580
DDVISTMRRP SLKDEVYVVG ADYSWGLISS WIEGALETSE NVINDYFL
```

In one embodiment, the fusion protein is zHER2:342(K4R/K7A/K27R/K49R/K50A/K58A)-APIT.

zHER2:342(K4R/K7A/K27R/K49R/K50A/K58A)-APIT comprises or consists of the amino acid sequence of SEQ ID NO. 5:

```
SEQ ID NO 5: amino acid number: 589, molecular weight: 66036.77 g/mol
  1 10 20 30 40 50 60
MVDNRFNAEMR NAYWEIALLP NLNNQQRRAF IRSLYDDPSQ SANLLAEARA LNDAQAPAGG
                70 80 90 100 110 120
GSGGGDGGGS MDGVSRNRRQ CNREVCGSTY DVAVVGAGPG GANSAYMLRD SGLDIAVFEY
              130 140 150 160 170 180
SDRVGGRLFT YQLPNTPDVN LEIGGMRFIE GAMHRLWRVI SELGLTPKVF KEGFGKEGRQ
              190 200 210 220 230 240
RFYLRGQSLT KKQVKSGDVP YDLSPEEKEN QGNLVEYYLE KLTGLQLNGE PLKREVALKL
              250 260 270 280 290 300
TVPDGRFLYD LSFDEAMDLV ASPEGKEFTR DTHVFTGEVT LDASAVSLFD DHLGEDYYGS
              310 320 330 340 350 360
EIYTLKEGLS SVPQGLLQAF LDAADSNEFY PNSHLKALRR KTNGQYVLYF EPTTSKDGQT
              370 380 390 400 410 420
TINYLEPLQV VCAQRVILAM PVYALNQLDW NQLRNDRATQ AYAAVRPIPA SKVFMTFDQP
              430 440 450 460 470 480
WWLENERKSW VTKSDALFSQ MYDWQKSEAS GDYILIASYA DGLKAQYLRE LKNQGEDIPG
              490 500 510 520 530 540
SDPGYNQVTE PLKDTILDHL TEAYGVERDS IPEPVTAASQ FWTDYPFGCG WITWRAGFHF
              550 560 570 580
DDVISTMRRP SLKDEVYVVG ADYSWGLISS WIEGALETSE NVINDYFL
```

The fusion protein zHER2:342(K4R/K7A/K27R/K49R/K50A/K58A)-APIT is highly suitable to be further modified, such as by PEGylation.

In a preferred embodiment, the active pharmaceutical ingredient (1) is modified, such as to increase its circulation time, enhance its serum half-life time, benefit the stability and/or increase enzyme activity of e.g. APIT.

Thereby, the targeting sequence can be modified and/or the protein (such as APIT) can comprise the modification.

A preferred embodiment for said modification is PEGylation, preferably with PEG sizes of about 1 to 40 kDa, more preferably up to 5 kDa.

Further suitable modification(s) are modification with poly(glycerols), poly(oxazolines), poly(amino acids), poly(acrylamides), poly(vinylpyrrolidones), and poly(zwitterions) and other modifications which, for example, benefit the stability and/or serum half-life time.

In one embodiment, the active pharmaceutical ingredient (1) is attached to a nanoparticle or the active pharmaceutical ingredient (1) is packed into/comprised within nanoparticles, such as lipid nanoparticles, liposomes or targeted nanoparticles.

### Active Pharmaceutical Ingredient (2)—Prodrug

The active pharmaceutical ingredient (2) of the kit of the present invention is a prodrug or proreporter comprising a compound of formula I or formula II.

A “prodrug” as used herein refers to a compound which comprises a drug which is inactive as long as it is covalently attached to the compound but is active after its release. The drug will have its effect or activity after release.

A “proreporter” as used herein refers to a compound which comprises a reporter or imaging sensor which is inactive as long as it is covalently attached to the compound but is active after its release. When the reporter is, for example, a fluorescence dye, it will emit specific fluorescence only after release.

The compound of formula I or formula II

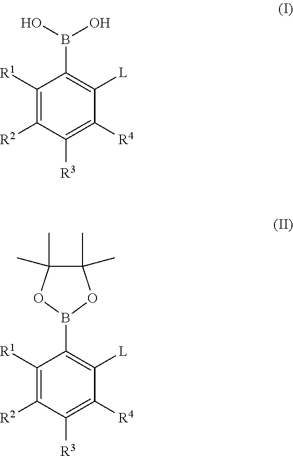

is a compound wherein
R¹ to R⁴ are each independently selected from H, OR⁵, NHR⁵, NHC(O)R⁵, SR⁵, halogen, C₁-C₆ alkyl, C₁-C₆ alkoxy, aryl, and C₁-C₂₀ alkyloxycarbonyl,
R⁵ is selected from H, C₁-C₆ alkyl, C₁-C₆ alkoxy, and C₁-C₆ alkyl or C₁-C₆ alkoxy substituted with a functional group for attachment of a polymer, peptide or protein,
L is selected from —(CH₂)₂—C(O)O—X and —CH═CH—C(O)O—X, and
X is a drug or a reporter,
or a pharmaceutically acceptable salt thereof.

In a preferred embodiment, X is a drug.

Preferably, the drug is an anti-cancer drug, an anti-viral drug and/or an antibiotic.

Examples for anti-cancer drugs are camptothecin, doxorubicin, gemcitabine, and cytarabine.

Examples for anti-viral drugs are 3′-hydroxyl nucleosides, ribavirin, ganciclovir, vidarabine, acyclovir, penciclovir, and zalcitabine.

Examples for antibiotics are aminoglycosides, streptomycin, chloramphenicol, tetracycline, macrolides, and geldanamycin.

In a preferred embodiment, X is a reporter or an imaging sensor.

Preferably, the reporter or imaging sensor is a fluorescence dye, a mass reporter/sensor or a chemoluminescence dye.

Examples for fluorescence dyes are near infrared (NIR) dyes, such as resorufin or 2-[2-(2,3-Dihydro-6-hydroxy-1H-xanthen-3-yl)ethenyl]-1-ethyl-3,3-dimethyl-3H-indolium.

Examples for mass reporters/sensors are peptides and small molecules, which are isotopic labelled.

In one embodiment, where X is a drug, the compound of the present invention is a compound of formula Ia, Ib, IIa or IIb:

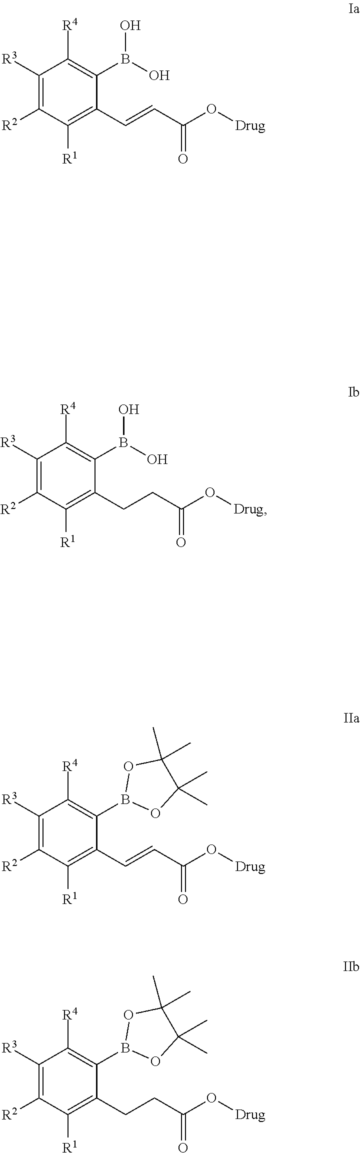

In embodiments of the active pharmaceutical ingredient (2) where X is a drug and said drug is camptothecin and the compounds are a compound of formula IIa or IIb:

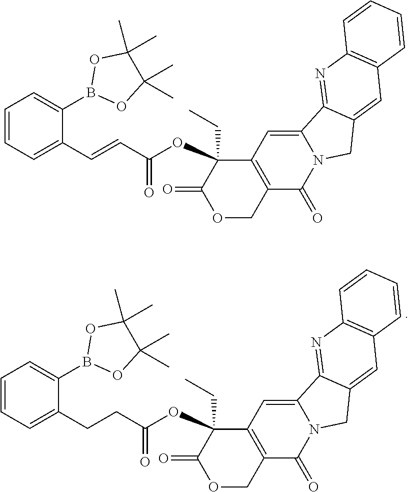 [SMILES: CC[C@@]1(OC(=O)/C=C/c2ccccc2B2OC(C)(C)C(C)(C)O2)C(=O)OCc2c1cc1n(c2=O)Cc2cc3ccccc3nc2-1 ; CC[C@@]1(OC(=O)CCc2ccccc2B2OC(C)(C)C(C)(C)O2)C(=O)OCc2c1cc1n(c2=O)Cc2cc3ccccc3nc2-1]

In a preferred embodiment,
R¹, R³ and R⁴ are each H, and
R² is selected from OR⁵, NHR⁵, NHC(O)R⁵, SR, halogen, C₁-C₆ alkyl, C₁-C₆ alkoxy (such as OMe), aryl, and C₁-C₂₀ alkyloxycarbonyl.

In one embodiment,
R¹, R³ and R⁴ are each H,
R² is OR⁵, NHR⁵, NHC(O)R⁵ or SR⁵, and
R⁵ is selected from H, C₁-C₆ alkyl, C₁-C₆ alkoxy, and C₁-C₆ alkyl or C₁-C₆ alkoxy substituted with a reactive group for attachment of a polymer, peptide or protein.

Said reactive group for attachment of a polymer, peptide or protein is preferably selected from SH, maleimide, methacrylate, furan, an ene-compound, and a click substituent.

Examples for ene-compounds are alkenes, such as C₁-C₆ alkene.

Examples for click substituents are azide, alkyne, substituents comprising dibenzocyclooctyne (DBCO) group, substituents comprising trans-cycloctene (TCO) group, tetrazine.

For example, compounds where R² is NHR⁵ and R⁵ is H:

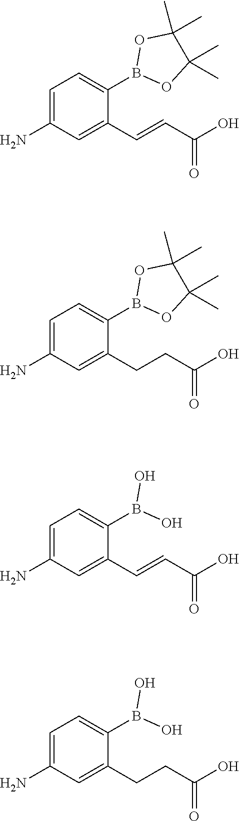 [SMILES: CC1(C)OB(c2ccc(N)cc2/C=C/C(=O)O)OC1(C)C ; CC1(C)OB(c2ccc(N)cc2CCC(=O)O)OC1(C)C ; Nc1ccc(B(O)O)c(/C=C/C(=O)O)c1 ; Nc1ccc(B(O)O)c(CCC(=O)O)c1]

In one embodiment, the active pharmaceutical ingredient (2) comprises one or more further moieties.

Said one or more further moieties can serve in improving bioavailability.

A preferred further moiety for improving bioavailability is a polymer.

Said polymer can be covalently attached to the compound of formula I or II at any of Ri to R⁴, preferably position R² wherein R² is preferably OR⁵ or NHR⁵ or SR⁵, and R⁵ is H.

An example is PEGylation, preferably with PEG sizes of about 1 to 40 kDa, more preferably up to 5 kDa.

Further examples are poly(glycerols), poly(oxazolines), poly(amino acids), poly(acrylamides), poly(vinylpyrrolidones), and poly(zwitterions).

In one embodiment, said further moiety is polyethylene glycol (PEG) which is covalently attached to the compound of formula I or II at any of R¹ to R⁴, preferably position R² wherein R² is more preferably OR⁵ or NHR⁵ or and R⁵ is H.

Said one or more further moieties can serve in improving selectivity.

Examples are peptides and proteins, such as tumor markers.

In one embodiment, said one further moiety is a peptide or protein which is covalently attached to the compound of formula I or II at any of R¹ to R⁴, preferably position R² wherein R² is preferably OR⁵, NHR⁵ or NHC(O) R⁵ and R⁵ is selected from C₁-C₆ alkyl or C₁-C₆ alkoxy substituted with a reactive group for attachment of a peptide or protein.

Said reactive group for attachment of a peptide or protein is preferably selected from SH, maleimide, methacrylate, furan, an ene-compound, and a click substituent, as discussed above.

Said reactive group is, for example, maleimide.

An example of a respective compound is the following compound:

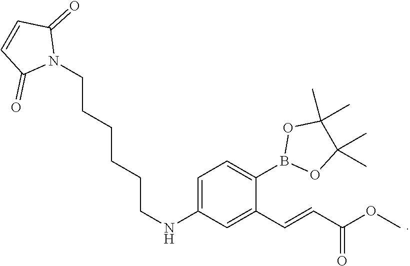 [SMILES: COC(=O)/C=C/c1cc(NCCCCCCN2C(=O)C=CC2=O)ccc1B1OC(C)(C)C(C)(C)O1]

In this compound R² is NHC(O)R⁵ and R⁵ is pentyl (C₅ alkyl) which is substituted with a reactive group, wherein said reactive group is maleimide.

The significantly increased “ROS level” (for example, the amount of hydrogen peroxide (H₂O₂)) at the site of action, which is caused by the active pharmaceutical ingredient (1), is used to release the drug or reporter from the active pharmaceutical ingredient (2) at the site of action.

In one embodiment, the active pharmaceutical ingredient (1) is provided for administration before administration of the active pharmaceutical ingredient (2).

In one embodiment, the active pharmaceutical ingredient (1) and/or the active pharmaceutical ingredient (2) is provided for oral, intravenous and/or intratumoral administration.

In one embodiment, the active pharmaceutical ingredient (1) is provided for intravenous or intratumoral administration; and the active pharmaceutical ingredient (2) is provided for oral, intravenous and/or intratumoral administration.

In a preferred embodiment, the active pharmaceutical ingredient (1) is provided for intravenous administration; and the active pharmaceutical ingredient (2) is provided for oral administration, such as a tablet.

### Medical Uses of the Kit

As outlined above, the present invention provides the kit of the present invention for use in the diagnosis, prevention and/or treatment of proliferative diseases, viral infections and/or non-viral infections.

The “treatment” as used herein also comprises the alleviation of the treated disease.

In one embodiment, where the kit of the present invention is for use in the diagnosis of proliferative diseases, viral infections and/or non-viral infections, the active pharmaceutical ingredient (2) is preferably a proreporter, i.e. a compound of formula I wherein X is a reporter.

In one embodiment, where the kit of the present invention is for use in the prevention and/or treatment of proliferative diseases, viral infections and/or non-viral infections, the active pharmaceutical ingredient (2) is preferably a prodrug, i.e. a compound of formula I wherein X is a drug.

The proliferative disease is preferably cancer, more preferably a cancer with solid tumor(s).

The cancer is preferably selected from, but not limited to, lung cancer, multi drug resistant lung cancer, head and neck cancer, breast cancer, prostate cancer, colon cancer, cervix cancer, uterus cancer, larynx cancer, gastric cancer, liver cancer, Ewing's sarcoma, acute lymphoid leukemia, acute and chronic myeloid leukemia, apoptosis resistant leukemia, pancreas cancer, kidney cancer, gliomas, melanomas, chronic lymphoid leukemia, and lymphoma.

In one embodiment, the cancer is a HER2-positive cancer.

Examples for a HER2-positive cancer are breast cancer and gastric cancer.

In one embodiment, the cancer is a cancer with metabolic auxotrophy on the amino acids L-lysine and/or L-arginine, such as hepatocellular carcinoma, malignant melanoma, malignant pleural mesothelioma, and prostate and kidney cancers.

The viral infection is preferably an infection with human immunodeficiency virus (HIV), hepatitis B virus (HBV) or/and hepatitis C virus (HCV).

The non-viral infection is preferably a microbial infection.

In a preferred embodiment, the active pharmaceutical ingredient (1) is provided for administration before administration of the active pharmaceutical ingredient (2).

In one embodiment, the active pharmaceutical ingredient (1) is administered first, in order to unfold its effect on the disease tissue (which comprises degradation of L-lysine, L-arginine and, above all, the production of H₂O₂). In addition to its therapeutic effect, the H₂O₂ produced is to be used as a differentiating release stimulus of the drug from the active pharmaceutical ingredient (2).

In one embodiment, the active pharmaceutical ingredient (1) and/or the active pharmaceutical ingredient (2) is provided for oral, intravenous and/or intratumoral administration.

In one embodiment, the active pharmaceutical ingredient (1) is provided for intravenous or intratumoral administration; and the active pharmaceutical ingredient (2) is provided for oral, intravenous and/or intratumoral administration.

In a preferred embodiment, the active pharmaceutical ingredient (1) is provided for intravenous administration; and the active pharmaceutical ingredient (2) is provided for oral administration, such as a tablet.

In one embodiment, after the administration of the active pharmaceutical ingredient (2) an irradiation is carried out.

In said embodiment, the prodrug comprises the compound of formula I or formular II, wherein L is —CH═CH—C(O)O—X, i.e. comprises a double bond in the linker between the phenylboronic acid part or the phenylboronic ester part and X. Due to the irradiation, such as with light having a wavelength of about 365 nm, there occurs an cis trans isomerization unfolding the internal cyclization of the cis structure of the phenylboronic acid part to coumarin triggered by ROS and at the same time the X, such as a drug, is released.

Coumarin or 2H-chromen-2-one or 2H-1-Benzopyran-2-one:

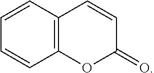 [SMILES: O=c1ccc2ccccc2o1]

Coumarin derivatives can be fluorescent, depending on the substituents R¹ to R⁴ allowing a detection of the drug release.

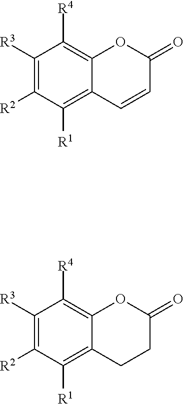

Method of Diagnosis and/or Treatment

As outlined above, the present invention provides a method for the diagnosis, prevention and/or treatment of a proliferative disease, a viral infection and/or a non-viral infection.

Said method comprises the administration of therapeutically effective amount of the kit of the present invention to a subject in need thereof.

In one embodiment, where the method of the present invention is for the diagnosis of proliferative diseases, viral infections and/or non-viral infections, the active pharmaceutical ingredient (2) is preferably a proreporter, i.e. a compound of formula I or formular II wherein X is a reporter.

In one embodiment, where the method of the present invention is for the prevention and/or treatment of proliferative diseases, viral infections and/or non-viral infections, the active pharmaceutical ingredient (2) is preferably a prodrug, i.e. a compound of formula I or formular II wherein X is a drug.

In one embodiment, the method comprises the administration of a therapeutically effective amount of the active pharmaceutical ingredient (1) of the kit of the present invention to a subject in need thereof, and the subsequent administration of a therapeutically effective amount of the active pharmaceutical ingredient (2) of the kit of the present invention to said subject.

A “therapeutically effective amount” of a kit of the present invention, or of the active pharmaceutical ingredient (1) and/or (2), refers to the amount which has to be administered to a subject in need thereof in order to achieve a desired therapeutic result or outcome. The skilled artisan will be able to determine said therapeutically effective amount and the suitable administration regimen.

The “treatment” as used herein also comprises the alleviation of the treated disease.

The proliferative disease is preferably cancer, more preferably a cancer with solid tumor(s).

The cancer is preferably selected from, but not limited to, lung cancer, multi drug resistant lung cancer, head and neck cancer, breast cancer, prostate cancer, colon cancer, cervix cancer, uterus cancer, larynx cancer, gastric cancer, liver cancer, Ewing's sarcoma, acute lymphoid leukemia, acute and chronic myeloid leukemia, apoptosis resistant leukemia, pancreas cancer, kidney cancer, gliomas, melanomas, chronic lymphoid leukemia, and lymphoma.

In one embodiment, the cancer is a HER2-positive cancer.

Examples for a HER2-positive cancer are breast cancer and gastric cancer.

In one embodiment, the cancer is a cancer with metabolic auxotrophy on the amino acids L-lysine and/or L-arginine, such as hepatocellular carcinoma, malignant melanoma, malignant pleural mesothelioma, and prostate and kidney cancers.

The viral infection is preferably an infection with human immunodeficiency virus (HIV), hepatitis B virus (HBV) or/and hepatitis C virus (HCV).

The non-viral infection is preferably a microbial infection.

In a preferred embodiment, the active pharmaceutical ingredient (1) is administered before the active pharmaceutical ingredient (2).

Preferably, between the administration of the active pharmaceutical ingredient (1) and the administration of the active pharmaceutical ingredient (2) is a time period of at least one hour up to several days, preferably one hour to one day.

In one embodiment, the method comprises an irradiation after administration of the active pharmaceutical ingredient (2). As discussed above, in this embodiment the prodrug comprises the compound of formula I or formular II, wherein L is —CH═CH—C(O)O—X.

Preferably, the active pharmaceutical ingredient (1) and/or the active pharmaceutical ingredient (2) are administered via for oral, intravenous and/or intratumoral administration.

In one embodiment, the active pharmaceutical ingredient (1) is administered via intravenous or intratumoral administration; and the active pharmaceutical ingredient (2) is administered via oral, intravenous and/or intratumoral administration.

In a preferred embodiment, the active pharmaceutical ingredient (1) is administered via intravenous administration; and the active pharmaceutical ingredient (2) is administered via oral administration, such as via a tablet.

### Preferred Embodiments

Herein, a novel treatment concept, which guarantees a selective increase of the ROS level in combination with targeted ROS-sensitive drug release (“prodrug” concept) by means of two modularly designed elements (pharmaceutically active ingredients) is described. See also FIG. 1 C.

The pharmaceutically active ingredient (1) has the task of selectively generating oxidative stress and the associated increase in ROS molecules at the site of action.

Said significantly increased “ROS level” (for example, the amount of hydrogen peroxide (H₂O₂)) at the site of action is then used to release the drug from the pharmaceutically active ingredient (2) through the increased ROS.

### —Pharmaceutically Active Ingredient (1)

An antitumor enzyme (Aplysia punctata ink toxin (APIT)) was discovered in the defensive secretion of the marine sea hare Aplysia punctata. It is a weakly glycosylated FAD-binding L-amino acid oxidase (FIG. 2 A) (Butzke et al, 2005).

The antitumor effect of APIT is based on the selective degradation of essential L-amino acids (L-lysine and L-arginine). This redox reaction produces reactive byproducts (H₂O₂ and NH₄⁺), which are also detrimental to the tumor. Selective degradation of amino acids L-lysine and L-arginine is suitable for therapy of cancers with metabolic auxotrophy on these amino acids, such as hepatocellular carcinoma, malignant melanoma, malignant pleural mesothelioma, and prostate and kidney cancers (Butzke et al., 2004). Since many tumor types lose the ability to produce arginine by converting citrulline in the urea cycle as they degenerate, the use of amino acid oxidases (for example, APIT) can be described as “starving” the cancer. In addition, reactive oxygen species (ROS) are produced during the enzyme reaction. It is known that tumors lose the ability to scavenge ROS (Perillo et al., 2020). Therefore, a very high level of ROS leads to cell death and thus tumor death, which is an additional aspect of APIT's efficacy.

APIT showed a very short blood circulation time (-3 h) in initial experiments. Modifications using polyethylene glycol (PEG) showed a significant increase in blood circulation time and an enzymatic activity of more than 48 h. This system is already under development for cancer therapy under the name Oncarex®, see also U.S. Pat. No. 8,759,060 B2.

Further development of APIT is the subject of the combination therapy described here. To increase the specificity of APIT, it can be modularly provided with different targeting sequences. The first protein conjugate tested was a zHER2:342-APIT (see FIG. 2 B and FIG. 4). The zHER2:342 sequence promotes selective binding to HER2-overexpressing (HER: human epidermal growth factor receptor) cells (Dong et al., 2016). This fusion protein was shown to bind to HER2-positive but not HER2-negative cells and to remain enzymatically active (see e.g. FIG. 5).

Furthermore, a previously undescribed lysine-deleted zHER2:342 mutant was synthesized as a fusion protein with APIT. The mutant was mutated to arginine in the case of functional properties of lysine such as surface charge or proximity to the HER2 binding site or to alanine in the case of structural properties of lysine. Starting from the native zHER2:342 sequence, the following mutations were made K4R/K7A/K27R/K49R/K50A/K58A. In silico stability studies show that the variant is more stable compared to the native zHER2:342 sequence. Again, the zHER2:342(K4R/K7A/K27R/K49R/K50A/K58A)-APIT fusion protein was shown to bind to HER2 positive but not HER2 negative cells and to be enzymatically active (see FIG. 5).

There is another reason why this variant zHER2:342 is advantageous. As described above, the half-life of APIT can be extended by PEGylation. In this case, APIT can be inserted via the cheap and efficient reductive alkylation or via N-hydroxysuccinimidyl-based chemistry. Always, the nucleophilic lysine residues of APIT as well as its N-terminus are the starting point in these syntheses. The result is a randomized PEGylation of APIT (somewhere randomly PEGylated), i.e., a heterogeneous product with mixtures of different APIT:PEG stoichiometries. The conventional zHER2 also has lysine residues, and these too would be PEGylated in the fusion protein. Preliminary studies show that this causes the zHER2 to lose its targeting properties to the tumor. The new zHER2:342, which no longer contains lysine residues but still finds its way to the tumor, can circumvent this problem.

The proteins APIT, the fusion protein zHER2:342-APIT and zHER2:342(K4R/K7A/K27R/K49R/K50A/K58A)-APIT were successfully recombinantly produced and purified. The proteins were verified for identity and purity by mass spectrometry and high-performance liquid chromatography (HPLC) (see FIGS. 3 and 4). Subsequently, both proteins were compared with respect to their enzymatic activity, in particular the production of ROS (H₂O₂) via a spectrofluorimetric experimental setup. The activity of both species is very similar:
APIT: 80 U/mg,
zHER2:342-APIT: 70 U/mg,
zHER2:342(K4R/K7A/K27R/K49R/K50A/K58A)-APIT: 70 U/mg.

The ability of the zHER2:342-APIT and zHER2:342(K4R/K7A/K27R/K49R/K50A/K58A)-APIT fusion proteins to bind to HER2-positive cells was confirmed in a cell culture experiment (see FIG. 5).

Important features of pharmaceutically active ingredient (1) are:
Fusion proteins of APIT and a targeting sequence, exemplified by zHER2:342-APIT, can be produced recombinantly.
Enzymatic activity and associated antitumor properties of the fusion protein are preserved.
Targeting ability of zHER2:342 is preserved.

### —Pharmaceutically Active Ingredient (2)

The pharmaceutically active ingredient (2) contains various phenylboronic acid derivatives (see e.g. FIG. 7A) or phenylboronic ester derivatives (FIG. 7B).

The drug release cascade is started by the ROS (especially H₂O₂) driven conversion of the boronic acid or the boronic ester to the phenol intermediate (see FIG. 7C). In the literature, the electronic pushing effect of the alcohol function is often used to release drugs in the para position (R² of formula I, II) (see Peiró Cadahía et al., 2019; Wang et al., 2016). In the pharmaceutically active ingredient (2) disclosed herein, drug release is achieved by intramolecular cyclization, namely attack of the alcohol function on the drug ester (Turner et al., 1987; Greenwald et al., 2000; Levine et al., 2012; Okoh et al., 2018; Walton and Dougherty, 2017). Thus, the R² position is still available for modifications.

See also Hankins et al., 2020 and WO 2021/262998 A1, wherein aryl boronate and aryl diaminoboryl compounds are described, which can be used for detecting the presence of hydrogen peroxide and other ROS in water, a cell or tissue.

The required ring closure can optionally be controlled by light-induced cis-trans isomerization of the double bond (shown with the dotted line in FIGS. 7A and B) (Turner et al., 1987; Gupta et al., 2022; Gagey et al., 2007). This dual controlled release mechanism ensures a controlled drug release and a coumarin derivative as a cyclization product is formed only at the site of action. As mentioned above, the aromatic provides additional opportunities for further functionalization (especially R²). On the one hand, this allows the spectroscopic properties of the resulting coumarin derivative to be modified (fluorescence for detection), but also other important modular modifications (protein, peptide and polymer conjugation).

In general, cyclization strategies and cis-trans control are well known. However, the combination with H₂O₂ selectivity in one construct is novel and very promising.

Important features of pharmaceutically active ingredient (2) are:
Activity/toxicity of the coupled drug is greatly reduced.
After release, efficacy is again fully guaranteed since no additional functionalities are present on the active ingredient at the end.
Novel combination of ROS sensitivity, intramolecular cyclization reaction and cis-trans isomerization in one structure.
Additional ring substitutions ensure the introduction of further structural motifs to improve bioavailability (polymer conjugation) and selectivity (tumor markers, proteins/peptides, zHER2:342).

### Embodiments of the Active Pharmaceutical Ingredient (2) with Camptothecin

a) Compounds 1, 2, 3 and 4 (as shown in FIG. 8A) were successfully synthesized at laboratory scale and analyzed by liquid chromatography with mass spectrometry coupling (LC-MS) (as shown in FIG. 8B). The schematic synthesis pathway is shown in FIGS. 8C and 8D. H₂O₂ sensitivity and, consequently, the drug-releasing cyclization mechanism by irradiation of compound 3 (λ=365 nm) and without irradiation of compound 4 was confirmed by HPLC (see FIGS. 9A, B, C and D).

In vitro experiments demonstrate significantly reduced cytotoxicity of compound 3 and compound 4 compared to camptothecin (FIGS. 9E and F). Only after ROS treatment in combination with irradiation, if necessary, is increased cytotoxicity again observed due to the release of camptothecin.
b) As discussed herein, additional functionalities are possible on the aromatic ring of the central linker unit.

Compounds 5, 6, 7 and 8 which are compounds with an amine functionality at R² (as shown in FIGS. 10A and 10B) were successfully synthesized.

This amine functionality can be used for further reactions. An example of an acid-amine coupling is compound 9, as shown in FIGS. 10C and 10D, where a maleimide functionality was introduced. Said maleimide functionality is often used in protein and polymer conjugation. This functionality can also be used to change the spectroscopic properties of the resulting coumarin derivative for analytical purposes.
c) Another aspect of the cyclization mechanism is the cis-trans conformation of the double formation as for example in compounds 1, 3, 5 and 7. The cis-trans conformation is controlled with light of specific wavelength, as shown for compound 5 in FIG. 11.

The following examples and drawings illustrate the present invention without, however, limiting the same thereto.

### BRIEF DESCRIPTION OF THE DRAWINGS

FIG. 1. Schematic representation of the principles of action.

Comparison of ROS levels in healthy tissue (blue bars) and diseased tissue (orange bars).

A) Selective modulation of ROS levels in diseased tissue

B) Natural ROS gradient as a release mechanism for ROS-sensitive “prodrugs”.

C) Combination of A and B to increase selectivity and efficacy.

FIG. 2. Aplysia punctata ink toxin (APIT).

A) Mechanism of APIT catalyzed enzyme reaction.

B) Schematic representation of the zHER2:342-APIT fusion protein. The zHER2:342 affinity domain is shown as a helical structure.

FIG. 3. Recombinant APIT
A) Analysis of the purified APIT by MALDI mass spectrometry (MALDI: matrix-assisted laser desorption ionization).

B) HPLC elution of the protein APIT.

FIG. 4. Fusion protein zHER2:342-APIT

A) Analysis of purified zHER2:342-APIT by MALDI mass spectrometry.

B) HPLC elution of the protein APIT.

FIG. 5. Selective binding of the fusion proteins to HER2.

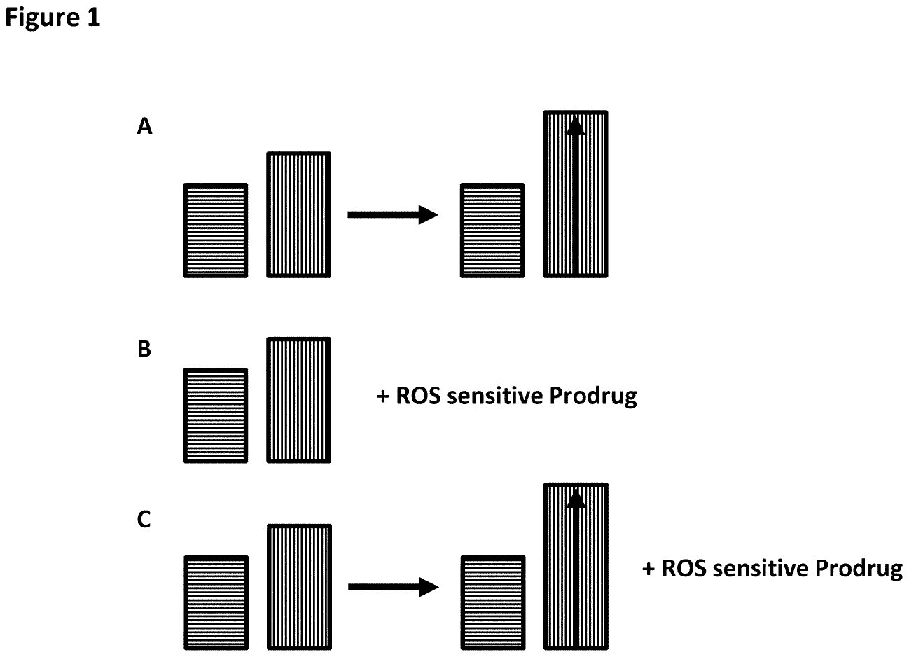

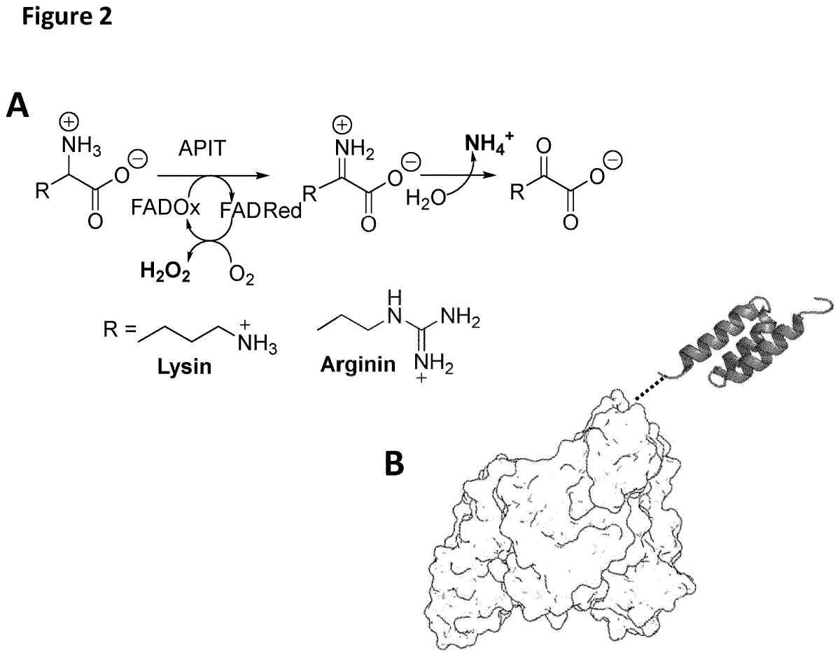

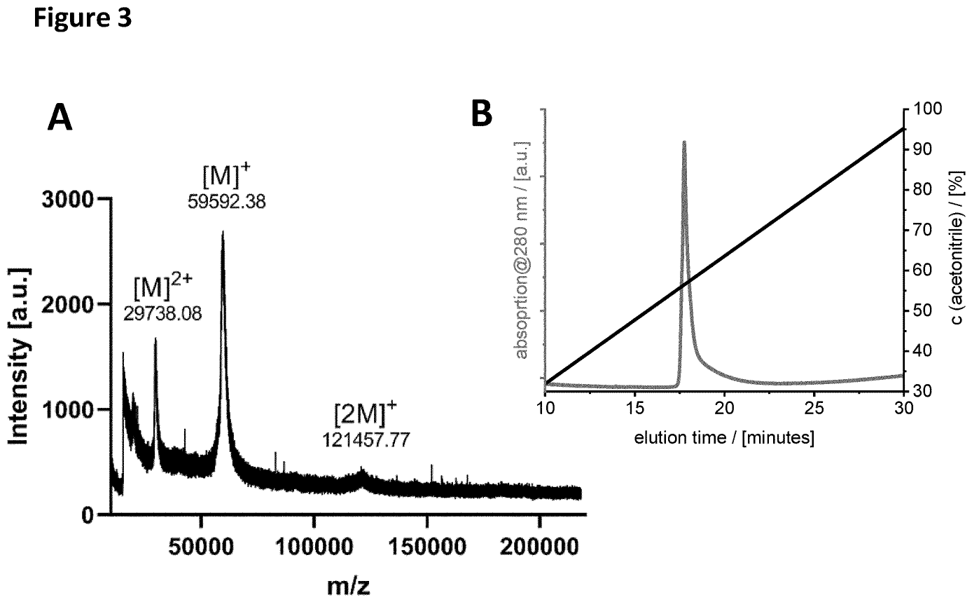

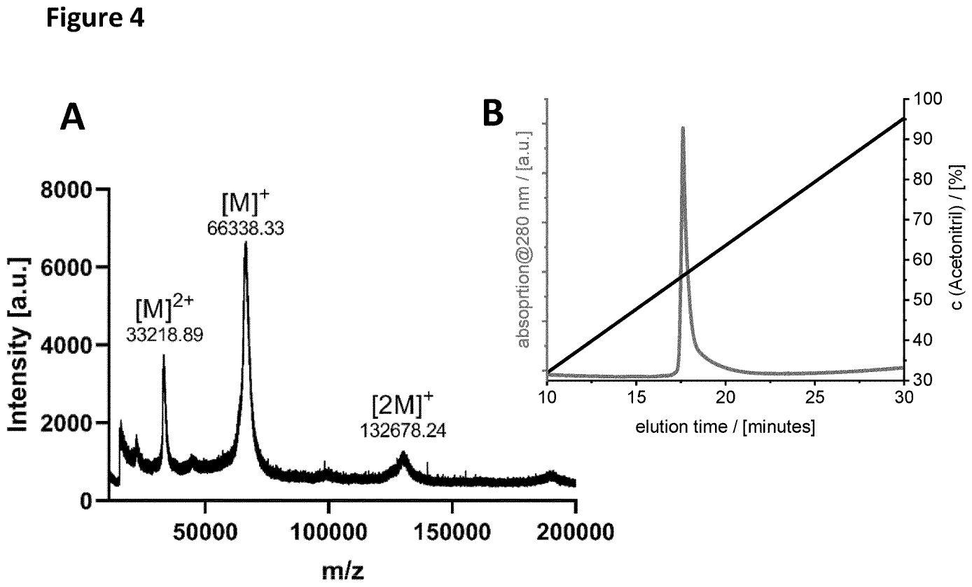

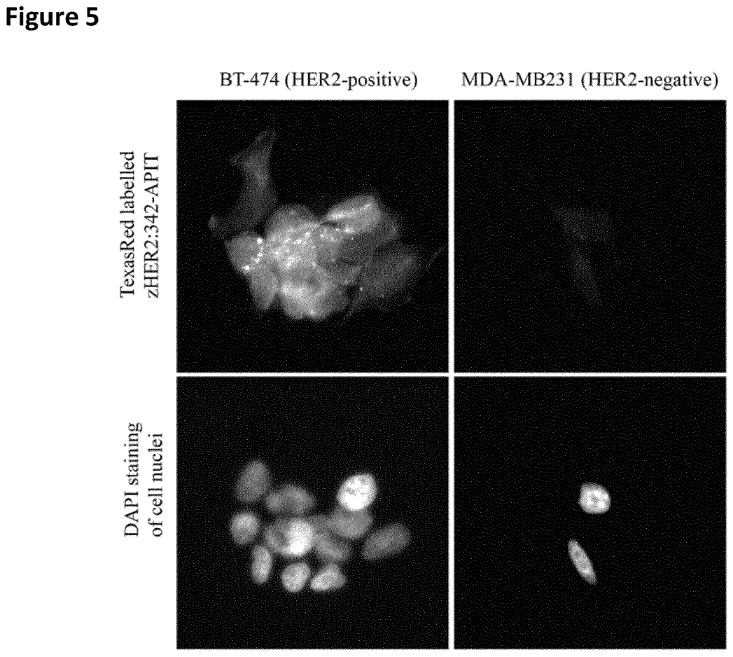

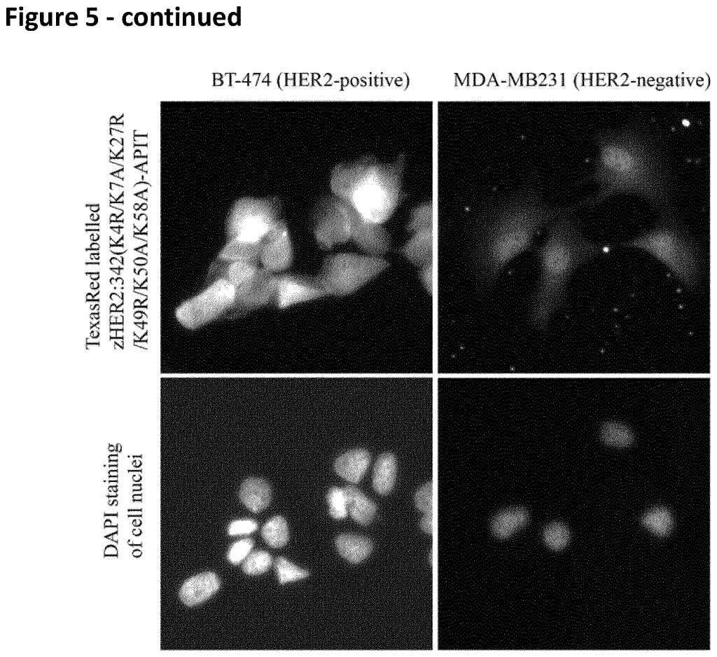

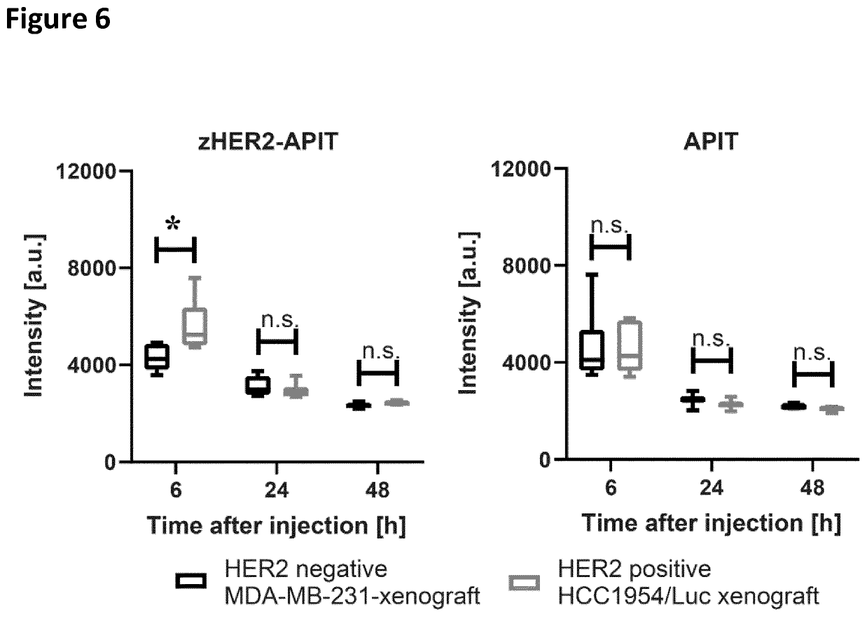

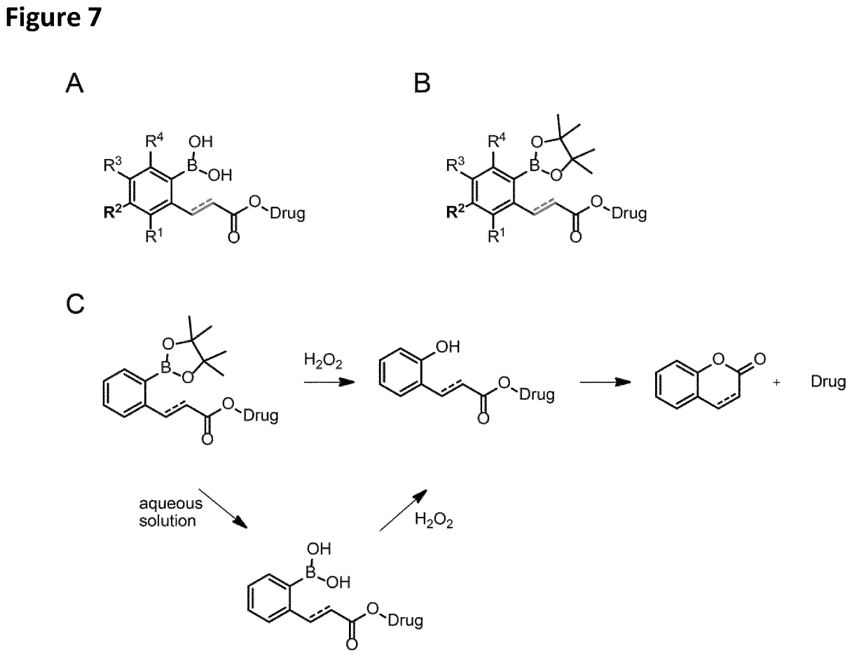

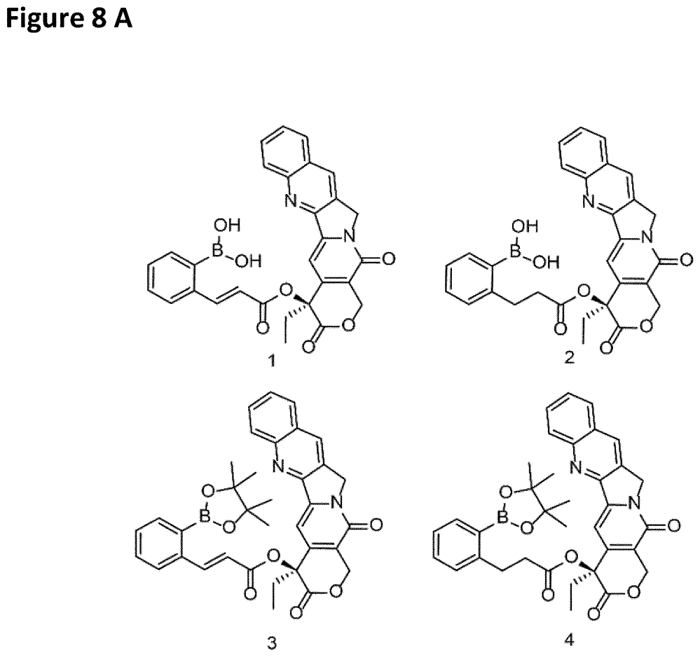

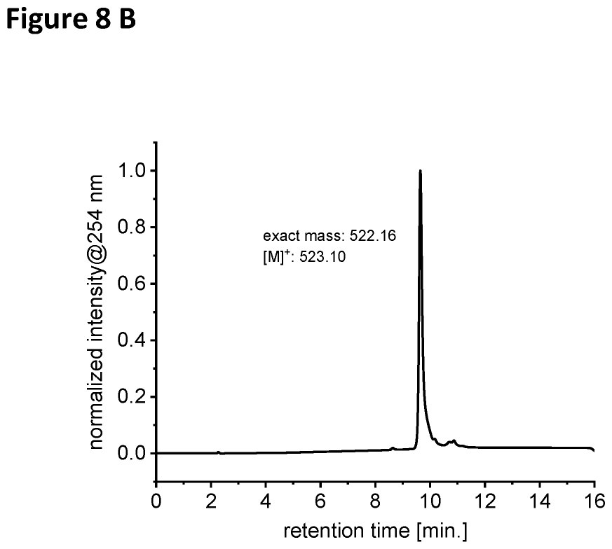

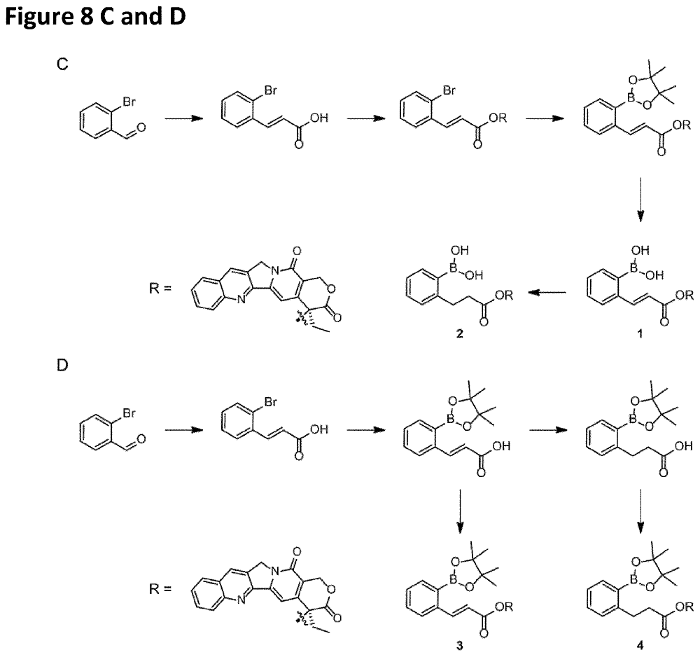

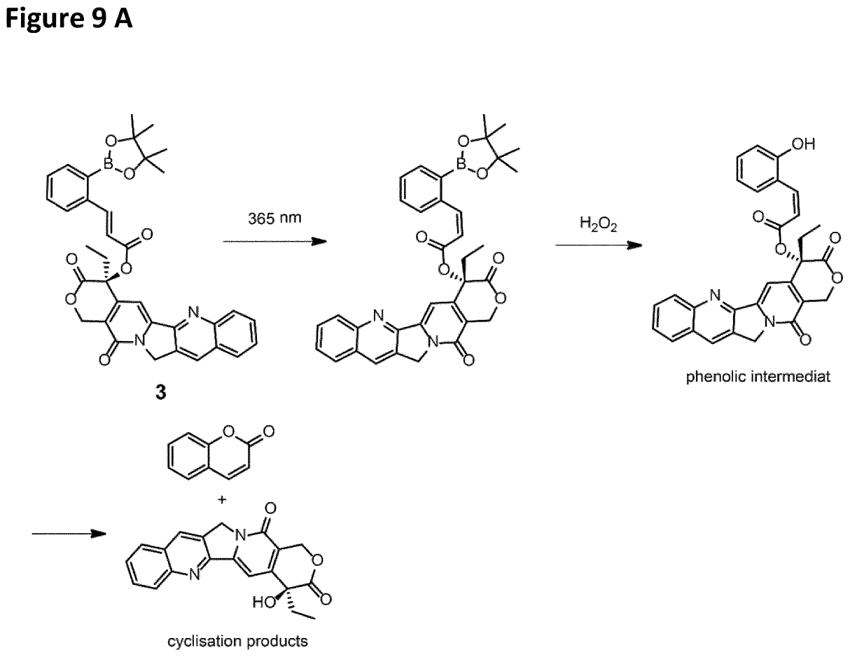

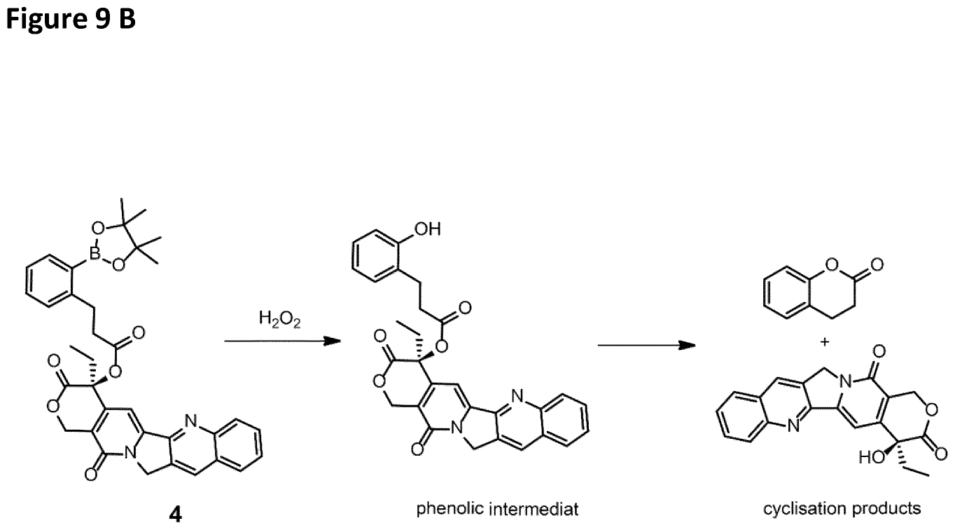

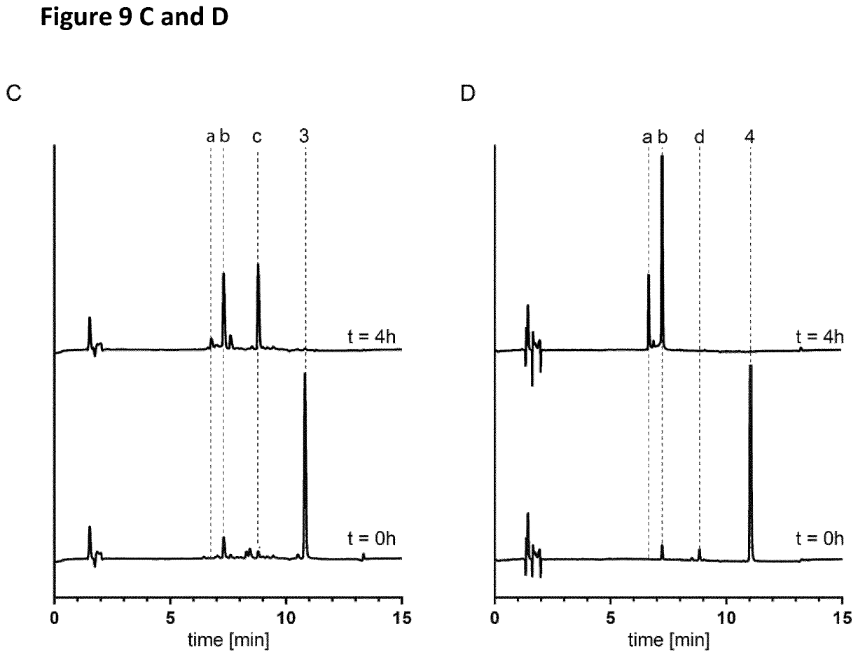

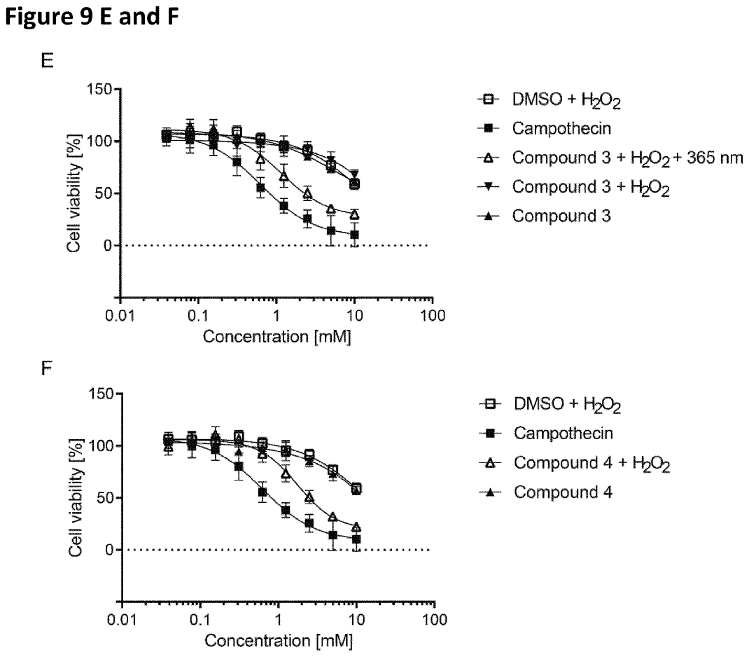

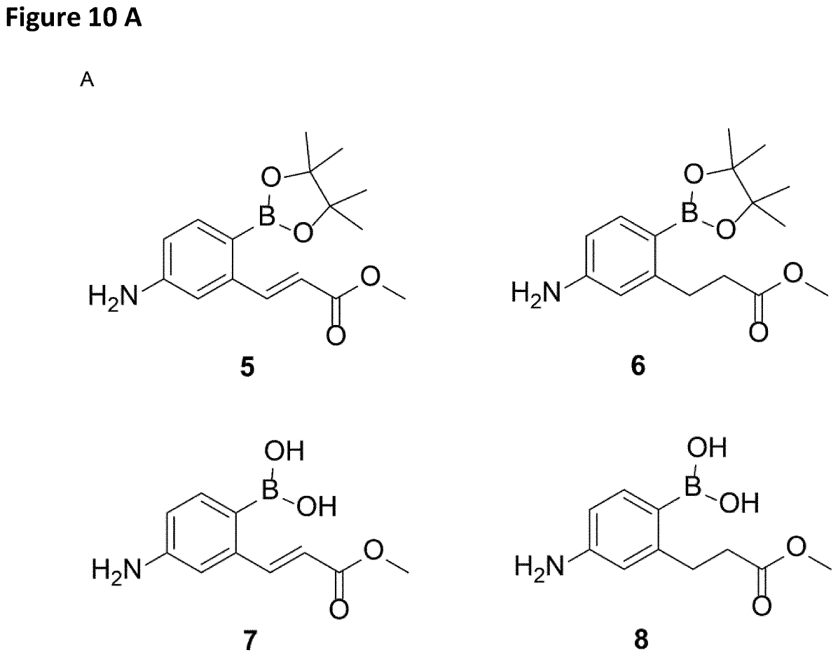

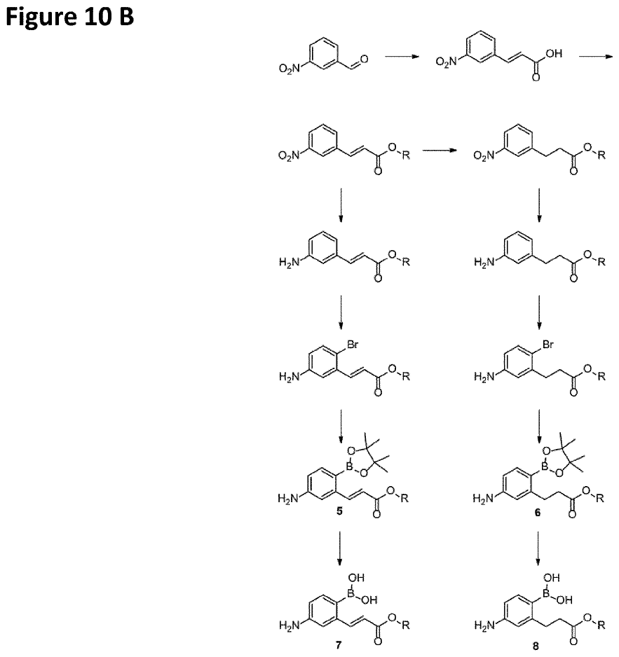

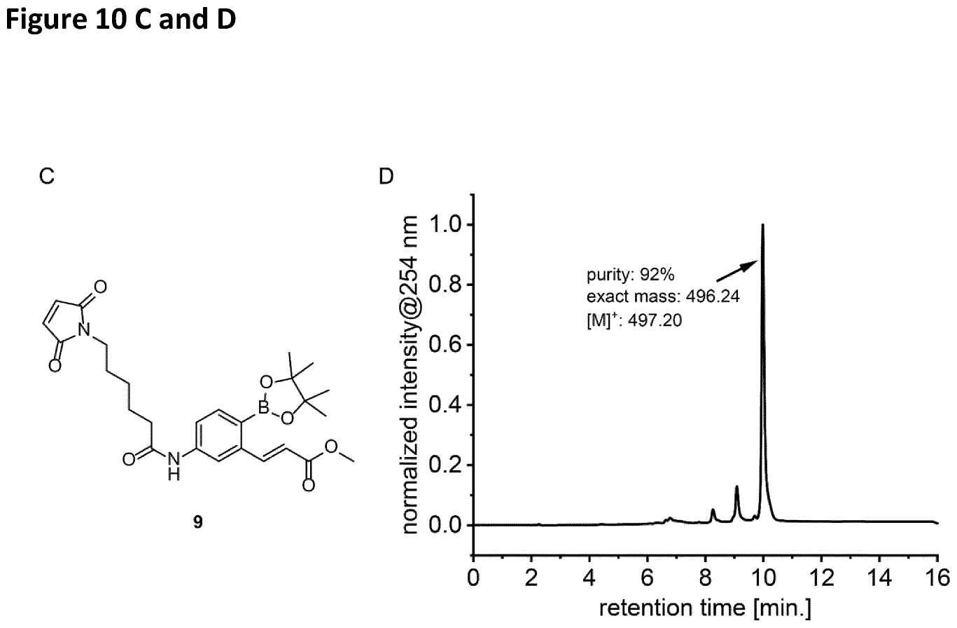

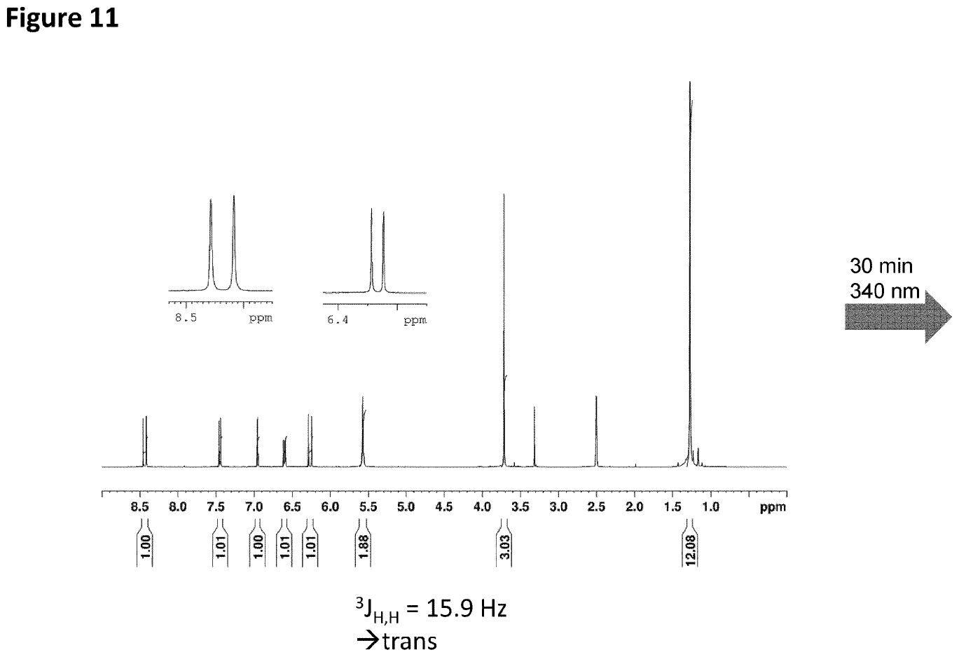

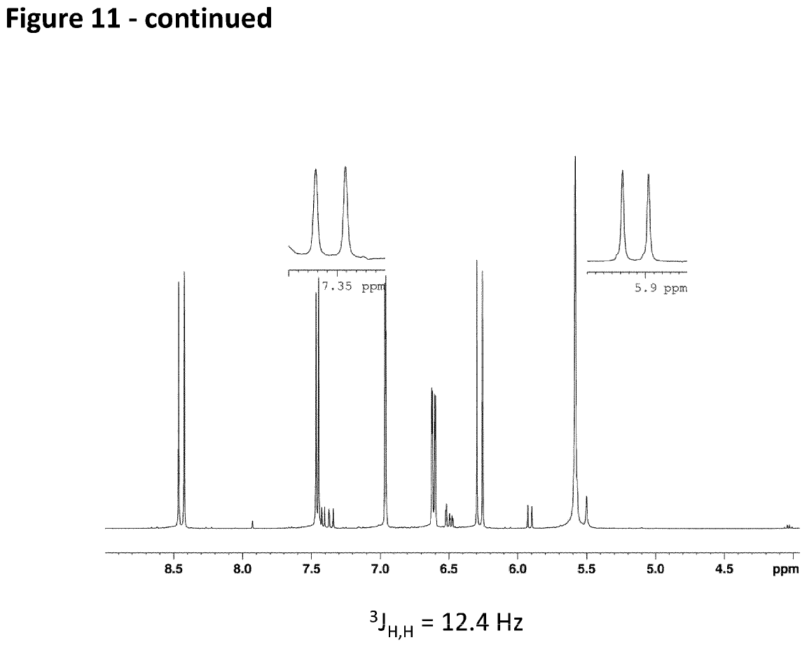

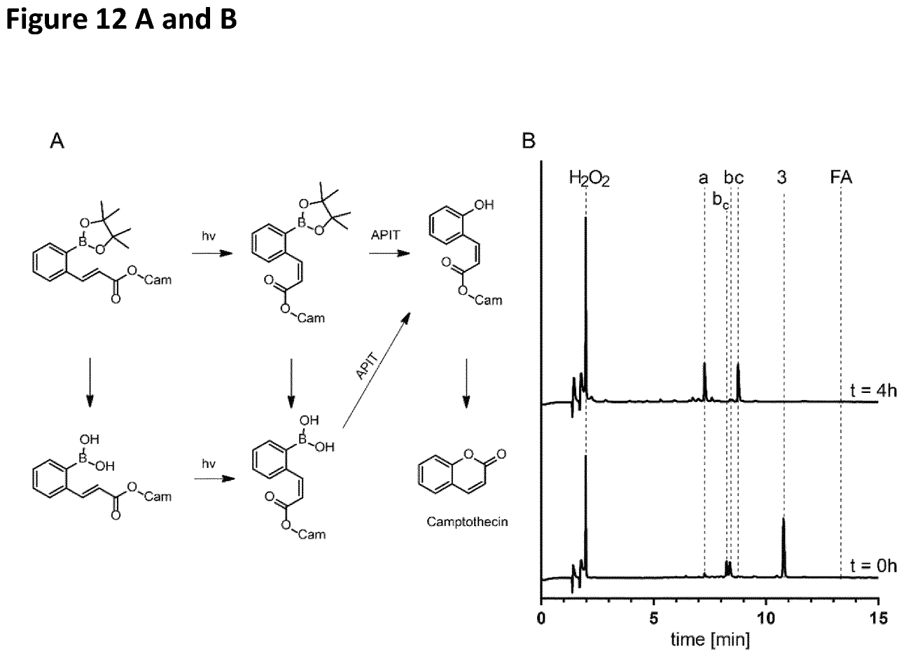

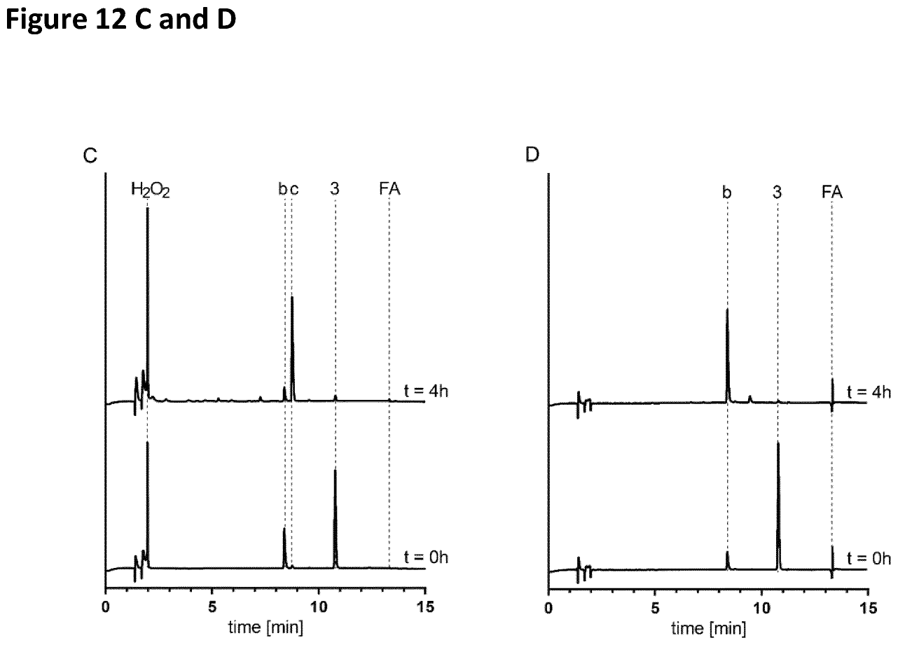

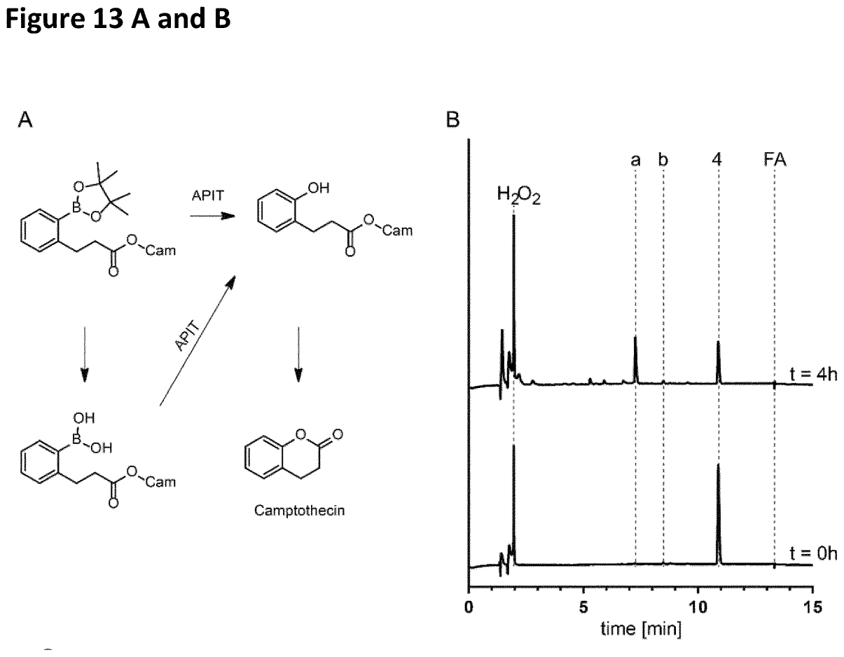

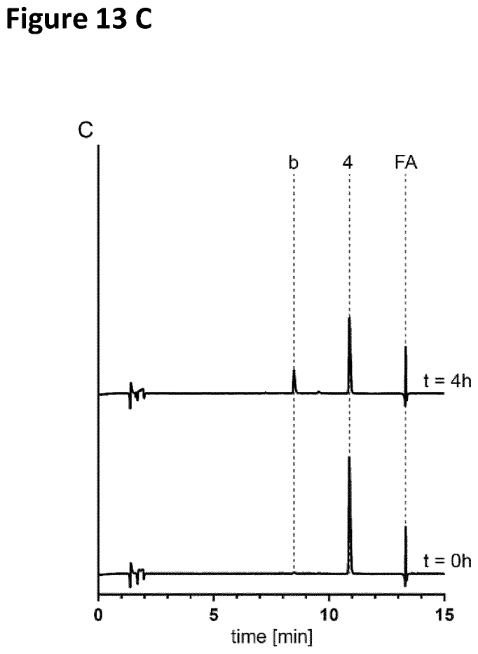

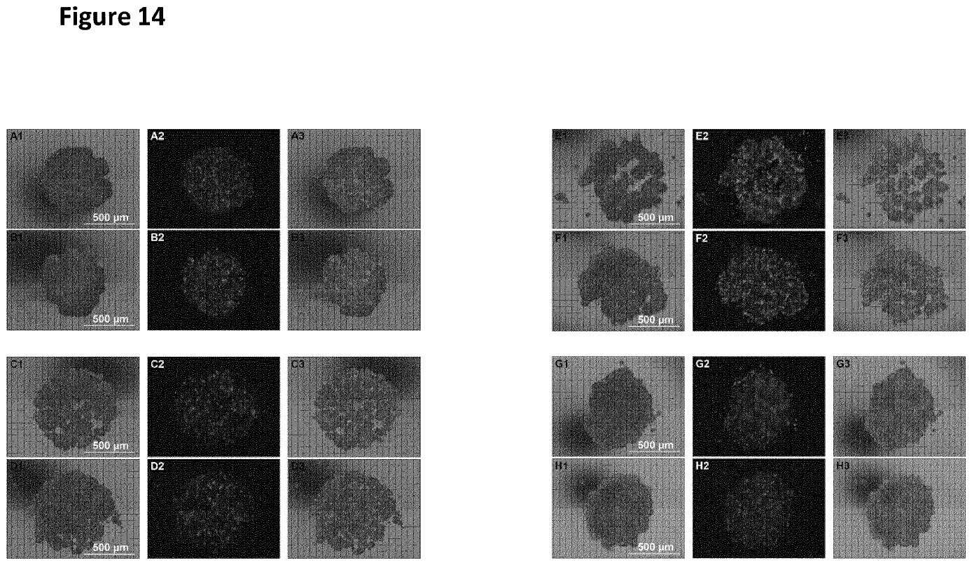

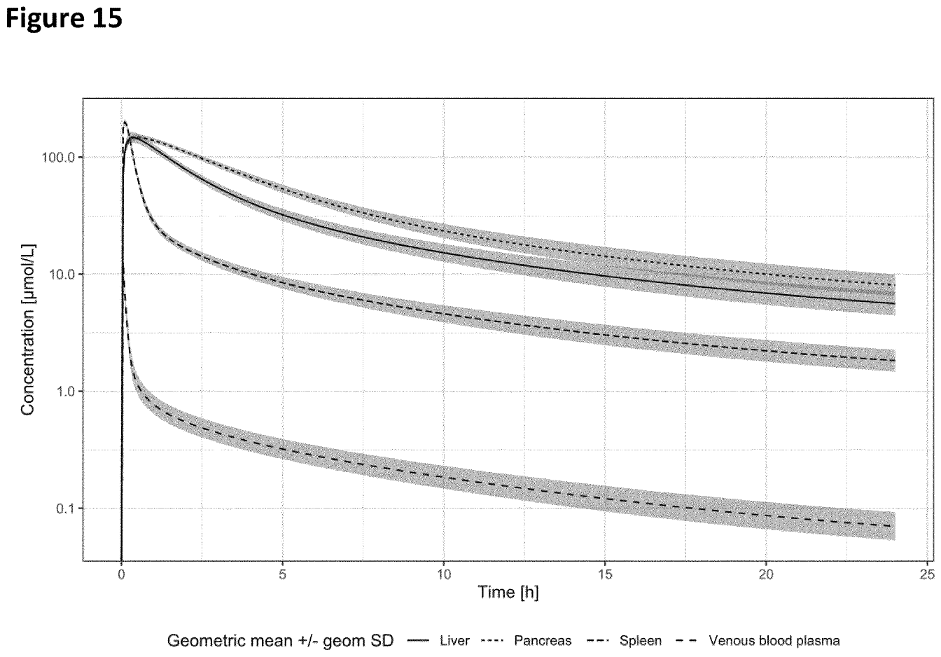

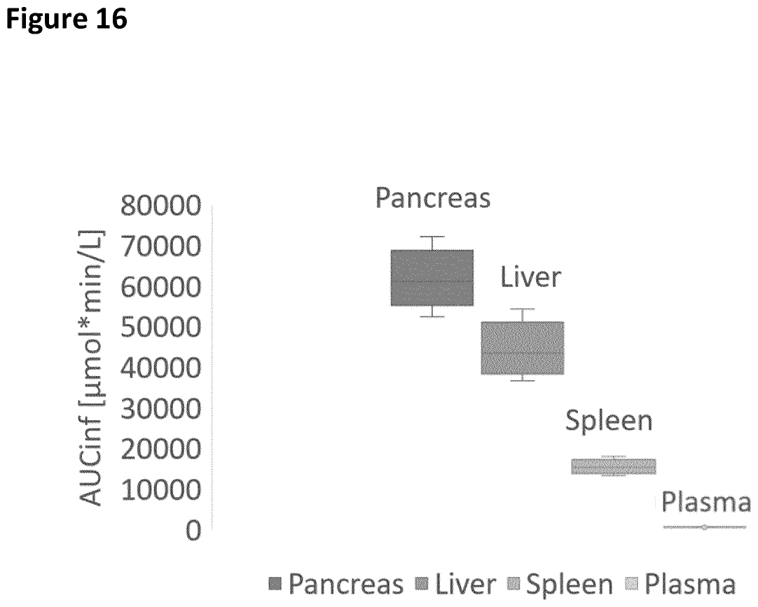

Shown are results of cell culture experiments to confirm the selective HER2 binding of the fusion proteins. The fusion proteins zHER2:342-APIT and zHER2:342(K4R/K7A/K27R/K49R/K50A/K58A)-APIT were labeled with fluorescent dye and probed for HER2 binding on fixed cells. Cell nuclei were stained with Hoechst33342.

Left panels: HER2-positive cells (BT474 cell line). Here, the fluorescent dye labeled fusion proteins bind to the HER2 receptor located on the cell membrane.

Right panels: HER2-negative cells (MDA-MB-231). The labeled fusion proteins do not bind specifically to the cell membrane (negative control).

FIG. 6. Tumor targeting and biodistribution.

Tumor targeting and biodistribution of (A) zHER2-APIT-NIR and (B) APIT-NIR in mice with HER2-negative tumor (black) and HER2-positive tumor (gray); (whiskers indicate 10-90% percentile values, line in the box is the median; significance was tested with the Mann-Whitney U test, *p<0.05 was considered statistically different; n.s.=not significant; n=6).

FIG. 7. Structure of component (2), the prodrug, and mechanism of drug release.

A) General structural formula of phenylboronic acid derivatives with coupled drug. Compounds with and without double bond (the respective double bond is shown with an interrupted line) are used.

B) General structural formula of phenylboronic ester derivatives with coupled drug. Compounds with and without double bond (the respective double bond is shown with an interrupted line) are used.

C) Drug release. In the first step, a conversion of phenylboronic ester or phenylboronic acid to phenol is carried out by means of H₂O₂, followed by drug release by means of intramolecular cyclization reaction.

FIG. 8. Embodiments of the active pharmaceutical ingredient (2) with camptothecin.

A) Structures of compounds 1, 2, 3 and 4.

B) LC-MS analysis of compound 1.

C) Example of a synthesis route of compounds 1 and 2.

D) Example of a synthesis route of compounds 3 and 4.

FIG. 9. Analysis of the drug release mechanism of one embodiment of the active pharmaceutical ingredient (2) with camptothecin and their activity on HEK cells.

A) Mechanism of drug release for compound 3: a phenolic intermediate is formed under the influence of H₂O₂, and then after cyclisation camptothecin is released, if the compound was irradiated with light of a wavelength of 365 nm bevor.

B) Mechanism of drug release for compound 4: a phenolic intermediate is formed under the influence of H₂O₂, and then after cyclisation camptothecin is released.

C) HPLC analysis of compound 3 after H₂O₂ treatment (a) oxidized camptothecin, (b) camptothecin (c) trans phenolic intermediate (3) compound 3.

D) HPLC analysis of compound 4 after H₂O₂ treatment (a) oxidized camptothecin, (b) camptothecin (d) phenylboronic acid of compound 4 (3) compound 4

E) WST-1 assay on HEK cells. Compound 3 (untreated, filled triangle) compared with camptothecin (filled squares), H₂O₂ treated compound 3 (upside down triangle) and irradiated and H₂O₂ treated compound 3 (triangle). As control cells were treated with DMSO and H₂O₂. All solutions were quenched with sodium pyruvate solution prior use.

F) WST-1 assay on HEK cells. Compound 4 (untreated, filled triangle) compared with camptothecin (filled squares), H₂O₂ treated compound 4 (triangle). As control cells were treated with DMSO and H₂O₂. All solutions were quenched with sodium pyruvate solution prior use.

FIG. 10. Embodiments of the active pharmaceutical ingredient (2) with a functionality at position R2.

A) Structures of compounds 5, 6, 7 and 8.

B) Example of a synthesis route of compounds 5, 6, 7 and 8.

C) Structure of compound 9, where a maleimide functionality was introduced.

D) LC-MS analysis of compound 9.

FIG. 11. Light-induced cis-trans isomerization.

of compound 5, as an example.

FIG. 12. Drug release of compound 3 in the presence of APIT

A) Mechanism of drug release: By irradiating the dissolved compound 3 with light at a wavelength of 365 nm, the double bond isomerises from trans to cis. By adding APIT to this solution, the boronic ester is converted to phenol, due to the hydrogen peroxide production of APIT. The active substance camptothecin is then released by an intramolecular ring closure. In the buffer used, part of the boronic ester is simultaneously hydrolysed to boronic acid. However, the addition of APIT also enables the formation of the phenol and thus the intramolecular ring closure and the release of the active ingredient.

B) Compound 3 treated with APIT and 100 mM Lysine after irradiation with light with a wavelength of 365 nm (a) camptothecin, (b_{c}) cis phenylboronic acid of compound 3, (b) trans phenylboronic acid of compound 3, (c) trans phenolic intermediate and (3) compound 3.

C) Compound 3 treated with APIT and 100 mM Lysine without irradiation. (b) trans phenylboronic acid of compound 3, (c) trans phenolic intermediate and (3) compound 3.

D) Compound 3 treated with 100 mM Lysine without irradiation (b) trans phenylboronic acid of compound 3, and (3) compound 3.

FIG. 13. Drug release of compound 4 in the presence of APIT

A) Mechanism of drug release: By adding APIT to a solution of compound 4, the boronic ester is converted to phenol, due to the hydrogen peroxide production of APIT. The active substance is then released by an intramolecular ring closure. In the buffer used, part of the boronic ester is simultaneously hydrolysed to boronic acid. However, the addition of APIT also enables the formation of the phenol and thus the intramolecular ring closure and the release of the active ingredient.

B) Compound 4 treated with APIT and 100 mM Lysine. (a) camptothecin, (b)phenylboronic acid of compound 4 and (4) compound 4.

C) Compound 4 treated with 100 mM Lysine (b) phenylboronic acid of compound 4 and (4) compound 4.

FIG. 14. Fluorescence imaging of spheroids undergoing various treatments.

Spheroids were subjected to growth medium (A, B), camptothecin treatment (C, D), activated Compound 4 (E, F), and inactive Compound 4 (G, H). Spheroid architecture was visualized using Phase contrast (1), while dead cells, stained with PI, are highlighted in red (2). The merged image (3) shows the structural details with the identification of dead cells.

FIG. 15.

Simulated concentration time profiles (geometric mean and geometric standard deviations) for a population of 100 adult individuals (age 18 to 65, BMI 18.5 to 25 kg/m²) receiving 350 mg/m² pf MRD 570 as i.v. bolus single dose.

FIG. 16.

Exposition (AUC_{0→inf}) of different organs after a single dose of 350 mg/m² compound 4 simulated for 100 adult individuals (age 18 to 65, BMI 18.5 to 25 kg/m²)

### EXAMPLES

### Example 1 Recombinant APIT and Fusion Proteins and their Selective Binding to Target Tumor Cells

### 1.1 Recombinant Expression of APIT/zHEr2APIT and Variants

Competent E. coli HMS174 were transformed with the pET26b-based plasmid with inserted APIT/zHER2APIT encoding gene between NdeI and EcoRI restriction sites. Expression was controlled by T7 promoter and induced by 1 mM IPTG (Sigma Aldrich, Steinheim, Germany) at an OD₆₀₀ of 0.8-1.0. After 4 h cells were harvested by centrifugation at 5000 g, 4° C. Expression resulted in inclusion bodies. Pelleted cells were resuspended in 50 mm Tris pH 8.0. Inclusion bodies were isolated in several sonification steps using a Bandelin Sonoplus ultrasonic device (Berlin, Germany).

### 1.2. Refolding

0.5 g of purified inclusion bodies were solubilized in 100 mL 6 m Urea, 50 mm Tris, 200 mm NaCl, 100 mm DTT at pH 8.0 overnight at 4° C. Solubilized inclusion bodies were centrifuged at 5000 g, 4° C. for 30 min and filtrated using a 0.2 μm PS-filter. The protein was diluted in refolding buffer 1 M Tris, 1 M NaCl, 1 mM EDTA, 25.6 μM FAD, 20 μM Cysteine, 0.5% PEG6000 pH 8.6 and stirred at 4° C. for 7 days.

### 1.3. Purification of APIT

The subsequent purification of refolded protein was performed in two steps using an anion exchange chromatography (HiTrap™ Q FF) and a size exclusion chromatography (HiLoad™ 16/600 Superdex™ 200 pg) from Cytiva (Uppsala, Sweden).

Elution was performed with buffer A (50 mm Tris, pH 8.0) and buffer B (50 mm Tris, 1 m NaCl, pH 8.0), starting with a washing step of 25% B in 10 CV, followed by an elution gradient of 25-60 in 10 CV. APIT was then loaded onto a HiLoad™ 16/600 Superdex 200 pg. Column was then run using PBS pH 7.4 at flowrate of 1 mL/min.

### 1.4. PEGylation Strategy

APIT, zHER2APIT or variant was dialyzed in PBS pH 7.4 overnight at 4° C. and concentration was adjusted to 1.1 mg/mL. 20 eq of PEG-NHS 5 kDa (Celares GmbH, Berlin, Germany) was dissolved at a concentration of 408 mg/mL in PBS pH 7.4 and 1:10 (v:v) added to APIT. The reaction was performed overnight at 4° C. continuously shaking. Reaction was purified using an anion exchange chromatography (HiTrap™ Q HP, Cytiva, Uppsala, Sweden) with buffer A (50 mm Tris, pH 8.0) and buffer B (50 mm Tris, 1 m NaCl, pH 8.0). The reaction was diluted 1:3 in buffer A, a maximum of 10 mg of protein was loaded onto 1 mL of resin at a flowrate of 0.5 mL/min. Afterwards a washing step at 0% B for at least 10 CV and elution gradient from 0-100% B was performed. Elution peak was pooled and dialyzed against PBS pH 7.4, concentration was adjusted to 2 mg/mL PEGylated APIT was filtered using a 0.2 μm PS filter and stored light protected.

### 1.5. Degree of PEGylation

The degree of PEGylation was assessed by determination of the mass increase measured by MALDI mass spectra with an ultrafleXtreme mass spectrometer (Bruker Daltonics, Bremen, Germany) equipped with a 355 nm smartbeam-II™ laser.

20-50 μg of protein were desalted using Sep-Pak Vac C18 cartridges. The recommended manufacturers instruction was followed. Bound protein was eluted using 80% acetonitrile in ddH₂O+0.1% TFA and subsequently lyophilized at −100° C., 0.001 mbar.

For external calibration Bruker Protein II standard (Bruker Daltonics #8207234) was mixed with sinapinic acid solution (saturated in a 1:2 mixture (v/v) of acetonitrile and 0.1% TFA).

Drops were measured on a stainless-steel target (MTP 384 target plate ground steel, Bruker Daltonics #8280784). The proteins were analyzed after double layer preparation. First a thin matrix layer of SA solution (saturated in ethanol) was applied onto the stainless-steel target, second the sample solution (4 mg/mL in a 1:1 mixture of acetonitrile and ddH2O) was mixed with sinapinic acid solution (saturated in a 1:2 mixture (v/v) of acetonitrile and 0.1% TFA) and dropped onto the first layer. Several thousand shots were accumulated for the spectrum.

1.6 Fluorescent Labelling of APIT and zHER2-APIT

Labelling of 1 eq protein was performed with either 6 eq of AlexaFluor680™-maleimide (NIR) or 10 eq of TexasRed-maleimide in PBS containing 50 mM TCEP pH 7.4 at a protein concentration of 1.0 mg/mL overnight. Reaction mixture was purified via HiTrap Q HP using 50 mM Tris pH 8.0 (A) and 50 mM Tris 1 M NaCl pH 8.0 (B) with following method: 30% B for 45 CV, 30-100% B in 30 CV. The eluent was dialyzed in PBS. The degree of label (DOL) was determined according to the manufacturer's instructions using extinctions coefficient of 1.539 mLmg-1 cm-1 for APIT.

### 1.7. Endotoxin Reduction

Endotoxin amount was tested using the EndoZyme II-kit. Endotoxin amount in one injection of 100 μL should not exceed 3.6 EU. For endotoxin reduction an anion-exchange chromatography was used. All chemicals were purchased in at least American chemical society (ACS) grade. All equipment required was either endotoxin reduced by incubation in or flushing with 1 M NaOH for at least 6 h or baked at 200° C. for at least 6 h. Protein was loaded at low conductivity and elution was performed accordingly using 50 mM Tris pH 8.0 (A) and 50 mM Tris 1 M NaCl pH 8.0 (B): 0% B for 15 CV, 0-100% B in 50 CV.

### 1.8. Cell-Based HER-2 Binding Assay

HER2 positive BT-474 (ACC 64), adherent cells were cultured in RPMI 1640 containing 20% (v/v) FBS, MEM non-essential amino acids, 1 mM Pyruvate, 10 μg/ml human insulin. HER2-negative MDA-MB-231 (ACC 732) adherent cells were cultured in Leibowitz' L15, 20% FBS. Cells were cultured from frozen stocks and passaged two times prior to the assay. Cells were incubated at 37° C., 5% CO2 humidified. At a confluency of 80% cell passaging was performed by trypsination using 0.25% (w/v) trypsin/0.2% (w/v) EDTA. Cells were seeded at 10 k cells/well on sterilized degreased 4%-gelatin coated cover slides (12 nm) in 24-well plates and grown over night in the incubator. Cells were washed with PBS and fixated with freshly made 4% (w/v) paraformaldehyde solution for 30 min. Afterwards, cells were washed with PBS and incubated with eighter 1:100 dilution of HER2 antibody or 10 μg/mL zHER2-APIT-TxR each in PBS pH 7.4 overnight at 4° C. protected from light. Afterwards, fixated cells were washed with PBS, the secondary anti-rabbit-AlexaFluor488 was added to the HER2-staining for 1 h and washed again. Cell nuclei were stained for 10 min using Hoechst33342. Cells were washed again and mounted using Mowiol-488-solution. Fluorescence signal was imaged using a ZEISS motorized Axio ZI observer fluorescence microscope. Images were analyzed and processed using Zeiss Zen Pro software.

1.9. Comparison APIT and zHER2-APIT

Each mouse had a HER2-positive and HER2-negative tumor. Significantly higher levels of zHER2-APIT were found in HER2-positive xenografts six hours after exposure compared to HER2-negative tissues (FIG. 6A). APIT-alone (without the zHER2 affibody) did not show differences in the biodistribution to HER2-positive and negative tumors (FIG. 6B).

### Example 2 Synthesis of Functionalized and H₂O₂-Sensitive Phenylboronic Acid Derivatives and H₂O₂-Induced Drug Release

### 2.1. Chemicals

All chemicals were purchased from Sigma Aldrich and used without further purification.

2.2. Synthesis of (E)-3-(2-(4,4,5,5 tetramethyl-1,3,2-dioxaborolan-2-yl)phenyl)acrylic acid

1 Equivalent of 2-bromobenzaldehyde and 1.3 equivalents of malonic acid were stirred in a mixture of piperdine and pyridine at 110° C. for 90 minutes. Water and HCl (37%) were added to the reaction mixture and the resulting precipitate was filtered off and washed with water.

1 Equivalent of the white crystalline solid, 0.05 equivalents of Pd(dppf)Cl₂, 1.5 equivalents of bis(pinacolato)diboron and 1.5 equivalents of potassium acetate in anhydrous 1,4-dioxane was heated to 80° C. by means of microwave irradiation and stirred for 5 hours. After addition of diethyl ether, the reaction mixture was filtered through a plug of cellite, washed with diethyl ether and concentrated in vacuo. The crude product was purified by column chromatography.

### 2.3. Release of the Active Ingredient Camptothecin in the Presence of APIT

In the following, the H₂O₂-induced release of the active substance camptothecin in the presence of the H₂O₂-producing enzyme APIT is shown using substances 3 and 4 (FIG. 12 and FIG. 13). Further examples showing the release only in the presence of H₂O₂ are shown in FIGS. 9C and 9D.

a) Release of Camptothecin from Compound 3

### Methods

Compound 3 was dissolved in DMSO and then irradiated for 1 h with light at a wavelength of 365 nm. The DMSO stock solution was diluted in PBS and the resulting solution was diluted again 1:1 with a 100 mM lysine solution (pH 7). APIT was added to this reaction solution and the reaction was observed for 4 hours using HPLC. To show that the release of the active ingredient camptothecin is only possible after isomerisation of the double bond, the experiment was also carried out without irradiation.

In addition, the stability of the compound was determined in the presence of lysine by carrying out the experiment without the addition of APIT.

### Results

The H₂O₂ produced by APIT in the presence of lysine is sufficient for the phenol derivative to be formed from the boric ester or boric acid. If the substance is also irradiated with light so the double bond isomerises from trans to cis, camptothecin is released through an intramolecular cyclisation reaction.

b) Release of Camptothecin from Compound 4

### Methods

A stock solution of compound 4 was diluted with PBS and the resulting solution was diluted again 1:1 with 100 mM lysine solution (pH 7). To this mixture APIT was added and the reaction was observed for 4 hours using HPLC. To proof that the release of camptothecin is only possible in the presence of APIT, the experiment was also carried out without addition of APIT to the solution.

### Results

As already shown for compound 3, APIT can produce enough H₂O₂ in the presence of lysine to form the phenol intermediate. In contrast to compound 3, this can now react further without irradiation, so that camptothecin is released as a result of the cyclisation reaction.

### Example 3: Spheroid Formation in the Presence of Compound 4

Example 3 illustrates the effect of camptothecin and compound 4 on the formation of spheroids. The results of this example show the influence of activated compound 4 (after activation with H₂O₂) on the formation of a round and solid spheroid compared to non-activated compound 4 and camptothecin in the appended FIG. 14.

### Methods

In this example, immortalized murine fibroblast NIH3T3 cells were specifically chosen for generating spheroids due to their ability to produce a consistently uniform, rounded, and tightly cohesive structure. These spheroids also exhibit the additional capability of forming an extracellular matrix on the outer surface. To achieve this, NIH3T3 cells (150,000 cells/mL) were seeded on non-tissue culture treated petri dish coverslips in 10 μL drops. Subsequently, they were treated with camptothecin and compound 4, the latter being activated both with and without H₂O₂, and were incubated upside down for 24 hours at 37° C. under 5% CO₂. Following the incubation period, the spheroids were stained with propidium iodide (0.003 μg per 10,000 cells) for 3 minutes at room temperature. The analysis of these spheroids was conducted using an Axio Observer.Z1 microscope equipped with an A-Plan 10×/0.25 Ph1 objective (Zeiss). The images were recorded with a phase-contrast channel, and a 43 DsRed Reflector (λₑₓ=538-562 nm λₑₘ=570-640 nm) with a mercury vapor short-arc lamp.

### Results

Treatment with camptothecin and compound 4 previously activated with H₂O₂ led to disintegration of spheroids after 24 hours compared to non-activated compound 4 and the control with growth medium (DMEM with heat-inactivated FBS (10%), penicillin G (100 U/mL) and streptomycin (100 μg/μL) (FIG. 14). Without being bound to a theory, these results indicate that contact with activated compound 4 leads to the disintegration of the spheroids/tumor formation.

### Example 4 Physiologically Based Pharmacokinetic (PBPK) Model of Compound 4

Intracellular concentrations of compound 4 were simulated using a physiologically based pharmacokinetic (PBPK) model in the PK-Sim® 11 Update 2 integrated framework. In accordance with the 3R (replacement, refinement, reduction) to reduce animal research, the in-silico model was used as a rationale basis for further development of the candidate. The ability to accumulate in tumor tissue characterized by fenestrated, leaky capillaries was of interest in the current investigations.

As the standard PBPK model implemented in PK-Sim® 11 Update 2 does not include a cancer compartment, physiological organs with known fenestrated blood vessels were used as a surrogate. The intracellular concentrations in pancreas, liver and spleen were compared to each other and to peripheral plasma concentrations.

As no in vivo data was available on the compound, the model was built on certain assumptions and calculated parameters (see Table 1). In brief, the compound was assumed to be poorly soluble with a high plasma protein binding to albumin. A moderate hepatic metabolism was included in the model as well as glomerular filtration which might be negligible given the high plasma protein binding.

Drug exposition was highest in the pancreas, which is known for the fenestration of blood vessels, necessary to fulfill its endocrine function. In less well perfused organs with less leaky capillaries the intracellular concentration was predicted to be much smaller (spleen). In conclusion, intracellular exposition in tumor tissue is likely to reach 20- to 100-fold that of the plasma (FIGS. 15 and 16).

In the simulation, the drug was administered as a single dose of 350 mg/m² as i.v. bolus dose. In order to visualize biologic variability on drug exposure, a population of 100 individuals (age 18 to 65, BMI 18.5 to 25 kg/m²) was created and treated in-silico.

TABLE 1
Input parameters and processes that were manually included in the PBPK model

| Input parameter | Value | Source |
|---|---|---|
| Lipophilicity | 4.88 | Calculated logP (ChemDraw) |
| Fraction unbound (fᵤ) | 0.02 | Prediction DruMAP ver.1.5* |
| Molecular weight | 604.47 g/mol | ChemDraw |
| Has halogens | No | ChemDraw |
| pKa | Neutral | ChemDraw |
| Solubility | 0.05 mg/L | Assumption |
| Renal clearance process | fᵤ x renal blood flow | |
| Hepatic clearance | 300 μL/min/mg | Prediction DruMAP ver.1.5* |
| | microsomal protein | (worst case scenario) |

*Drug Metabolism and pharmacokinetics Analysis Platform (https://adme.nibiohn.go.jp/accessed 2023 Oct. 30)

The features disclosed in the foregoing description, in the claims and/or in the accompanying drawings may, both separately and in any combination thereof, be material for realizing the invention in diverse forms thereof

### REFERENCES

Butzke, D., et al., Hydrogen peroxide produced by Aplysia ink toxin kills tumor cells independent of apoptosis via peroxiredoxin I sensitive pathways. Cell Death & Differentiation, 2004. 11(6): p. 608-617.
Butzke, D., et al., Cloning and biochemical characterization of APIT, a new 1-amino acid oxidase from Aplysia punctata. Toxicon, 2005. 46(5): p. 479-489.
Dong, D., et al., Human Serum Albumin and HER2-Binding Affibody Fusion Proteins for Targeted Delivery of Fatty Acid-Modified Molecules and Therapy. Molecular Pharmaceutics, 2016. 13(10): p. 3370-3380.
Exner, N., et al., Mitochondrial dysfunction in Parkinson's disease: molecular mechanisms and pathophysiological consequences. The EMBO Journal, 2012. 31(14): p. 3038-3062.
Gagey, N., et al., Two-Photon Uncaging with Fluorescence Reporting: Evaluation of the o-Hydroxycinnamic Platform. Journal of the American Chemical Society, 2007. 129(32): p. 9986-9998.
Greenwald, R. B., et al., Drug Delivery Systems Based on Trimethyl Lock Lactonization: Poly(ethylene glycol) Prodrugs of Amino-Containing Compounds. Journal of Medicinal Chemistry, 2000. 43(3): p. 475-487.
Gupta, A., A. Gautam, and P. K. Sasmal, Photoactivatable o-Hydroxycinnamic Platforms for Bioimaging and Therapeutic Release. Journal of Medicinal Chemistry, 2022.
Hankins R A, Suarez S I, Kalk M A, Green N M, Harty M N, Lukesh III J C, An innovative hydrogen peroxide-sensing scaffold and insight towards its potential as an ros-activated persulfide donor. Angew. Chem. Int. Ed., 2020. DOI: 10.1002/anie.202010530
Johri, A. and M. F. Beal, Antioxidants in Huntington's disease. Biochimica et Biophysica Acta (BBA)—Molecular Basis of Disease, 2012. 1822(5): p. 664-674.
Levine, M. N. and R. T. Raines, Trimethyl lock: a trigger for molecular release in chemistry, biology, and pharmacology. Chemical Science, 2012. 3(8): p. 2412-2420.
Mattson, M. P., Pathways towards and away from Alzheimer's disease. Nature, 2004. 430(7000): p. 631-639.
Okoh, O. A. and P. Klahn, Trimethyl Lock: A Multifunctional Molecular Tool for Drug Delivery, Cellular Imaging, and Stimuli-Responsive Materials. ChemBioChem, 2018. 19(16): p. 1668-1694.
Peiró Cadahia, J., et al., Prodrug strategies for targeted therapy triggered by reactive oxygen species. MedChemComm, 2019. 10(9): p. 1531-1549.
Peng, X and Gandhi V. ROS-activated anticancer prodrugs: a new strategy for tumor-specific damage. Therapeutic delivery, 2012 3(7): 823-833.
Perillo, B., et al., ROS in cancer therapy: the bright side of the moon. Experimental & Molecular Medicine, 2020. 52(2): p. 192-203.
Reczek, C. R. and N. S. Chandel, The Two Faces ofReactive Oxygen Species in Cancer. Annual Review of Cancer Biology, 2017. 1(1): p. 79-98.
Sies, H. and D. P. Jones, Reactive oxygen species (ROS) as pleiotropic physiological signaling agents. Nature Reviews Molecular Cell Biology, 2020. 21(7): p. 363-383.
Turner, A. D., et al., Photochemical activation of acylated.alpha. -thrombin. Journal of the American Chemical Society, 1987. 109(4): p. 1274-1275.
Walton, D. P. and D. A. Dougherty, A General Strategy for Visible-Light Decaging Based on the Quinone Trimethyl Lock. Journal of the American Chemical Society, 2017. 139(13): p. 4655-4658.
Wang, J., et al., Leveraging H2O2 Levels for Biomedical Applications. Advanced Biosystems, 2017. 1(9): p. 1700084.

1. A kit of active pharmaceutical ingredients comprising;
(1) a fusion component comprising an agent, which increases the level of reactive oxygen species (ROS), and a targeting sequence, and
(2) a prodrug or proreporter comprising a compound of formula I or formula II 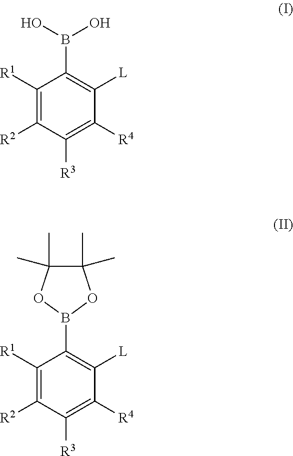
wherein
R¹ to R⁴ are each independently selected from H, OR⁵, NHR⁵, NHC(O)R⁵, SR^{I}, halogen, C₁-C₆ alkyl, C₁-C₆ alkoxy, aryl, and C₁-C₂₀ alkyloxycarbonyl,
R⁵ is selected from H, C₁-C₆ alkyl, C₁-C₆ alkoxy, and C₁-C₆ alkyl or C₁-C₆ alkoxy substituted with a reactive group for attachment of a polymer, peptide or protein,
L is selected from —(CH₂)₂—C(O)O—X and —CH═CH—C(O)O—X, and
X is a drug or a reporter,
or a pharmaceutically acceptable salt thereof.

2. The kit of claim 1, wherein the fusion component is a fusion protein and the agent, which increases the level of reactive oxygen species (ROS) is an L-amino acid oxidase,
and/or wherein the targeting sequence of active pharmaceutical ingredient (1) targets cancer cells, viruses, bacterial cells or cells of other microorganisms.

3. The kit of claim 1, wherein the targeting sequence of active pharmaceutical ingredient (1) targets human epidermal growth factor receptor (HER2)-overexpressing cells.

4. The kit of according to claim, wherein the active pharmaceutical ingredient (1) comprises a fusion protein having the amino acid sequence of SEQ ID NOs: 4 or 5.

5. The kit according to claim 1, wherein in the active pharmaceutical ingredient (2)
R¹, R³ and R⁴ are each H, and
R² is selected from OR⁵, NHR⁵, NHC(O) R⁵, SR⁵, halogen, C₁-C₆ alkyl, C₁-C₆ alkoxy, aryl and C₁-C₂₀ alkyloxycarbonyl.

6. The kit of claim 5, wherein R² is OR⁵, NHR⁵, NHC(O) R⁵ or SR⁵ and R⁵ is selected from H, C₁-C₆ alkyl, C₁-C₆ alkoxy, and C₁-C₆ alkyl or C₁-C₆ alkoxy substituted with a reactive group for attachment of a polymer, peptide or protein,
wherein said reactive group is selected from SH, maleimide, methacrylate, furan, an ene-compound, and a click substituent.

7. The kit according to claim 1, wherein X is a drug which is selected from
anti-cancer drugs,
anti-viral drugs; and/or
antibiotics;
or
a reporter which is selected from
fluorescence dyes, and
chemoluminescence dyes.

8. The kit according to claim 1, wherein the active pharmaceutical ingredient (2) comprises one or more further moieties,
wherein one further moiety is a polymer wherein said polymer is attached to the compound of formula I or II at any of R¹ to R⁴;
and/or
wherein one further moiety is a peptide or a protein,
wherein said peptide or protein is covalently attached to the compound of formula I or II at any of R¹ to R⁴.

9. The kit according to claim 1, wherein the active pharmaceutical ingredient (1) is provided for administration before administration of the active pharmaceutical ingredient (2), and/or
wherein the active pharmaceutical ingredient (1) and/or active pharmaceutical ingredient (2) are provided for oral, intravenous and/or intratumoral administration.

10. The kit of claim 1 comprising components for use in the diagnosis, prevention and/or treatment of proliferative diseases, viral infections and/or non-viral infections.

11. The kit for use according to claim 10, wherein the proliferative disease is cancer,
and/or wherein the viral infection is an infection with human immunodeficiency virus (HIV), hepatitis B virus (HBV) or/and hepatitis C virus (HCV),
and/or wherein the non-viral infection is a microbial infection.

12. The kit for use according to claim 10, wherein the active pharmaceutical ingredient (1) is provided for administration before the administration of active pharmaceutical ingredient (2), and/or
wherein the active pharmaceutical ingredient (1) and/or the active pharmaceutical ingredient (2) are administered via oral, intravenous and/or intratumoral administration.

13. The kit for use according to claim 10, comprising an irradiation after administration of the active pharmaceutical ingredient (2), preferably when L is —CH═CH—C(O)O—X.

14. A method for the diagnosis, prevention and/or treatment of a proliferative disease, a viral infection and/or a non-viral infection,
comprising the administration of therapeutically effective amounts of the kit according to claim 1, to a subject in need thereof.

15. The method of claim 14,
wherein the proliferative disease is cancer,
and/or wherein the viral infection is an infection with human immunodeficiency virus (HIV), hepatitis B virus (HBV) or/and hepatitis C virus (HCV),
and/or wherein the non-viral infection is a microbial infection.
and/or
wherein the active pharmaceutical ingredient (1) is administered before the active pharmaceutical ingredient (2),
and/or
wherein between the administration of active pharmaceutical ingredient (1) and active pharmaceutical ingredient (2) is a time period of at least one hour up to several days,
and/or
wherein the method comprises an irradiation after administration of the active pharmaceutical ingredient (2),
and/or
wherein the active pharmaceutical ingredient (1) and/or the active pharmaceutical ingredient (2) are administered via oral, intravenous and/or intratumoral administration.

16. The kit of claim 2, wherein the fusion component is a fusion protein and the agent, which increases the level of reactive oxygen species (ROS) is Aplysia punctata ink toxin (APIT) comprising the amino acid sequence of SEQ ID NO: 1.

17. The kit of claim 3, wherein the targeting sequence of active pharmaceutical ingredient (1) targets human epidermal growth factor receptor (HER2)-overexpressing cells, and is zHER2:342 comprising the amino acid sequence of SEQ ID NO: 2, or
a zHER2:342 variant wherein the lysine residues are deleted or substituted with arginine and/or alanine.

18. The kit of claim 8, wherein said peptide or protein is covalently attached to the compound of formula I or II at position R², wherein R² is OR⁵, NHR⁵ or NHC(O) R⁵ and R⁵ is selected from C₁-C₆ alkyl or C₁-C₆ alkoxy substituted with a reactive group for attachment of a peptide or protein being selected from SH, maleimide, methacrylate, furan, an ene-compound, and a click substituent.

19. The kit of claim 11, wherein the proliferative disease is a cancer with solid tumors.

20. The kit of claim 14, wherein the proliferative disease is a cancer with solid tumors.
